(19) United States
(12) Patent Application Publication
(10) Pub. No.: US 2013/0079237 A1
(43) Pub. Date: Mar. 28, 2013

(54) ALLERGEN MICROARRAY

(71) Applicants: Andrea Crisanti, London (GB); Tania Dottorini, Perugia (IT)

(72) Inventors: Andrea Crisanti, London (GB); Tania Dottorini, Perugia (IT)

(21) Appl. No.: 13/579,362

(22) PCT Filed: Feb. 16, 2011
(86) PCT No.: PCT/GB11/50302

§ 371 Date: Nov. 29, 2012

(30) Foreign Application Priority Data
Feb. 16, 2010 (GB) 1002601.1

(51) Int. Cl. G01N 33/68

(52) U.S. Cl. CPC G01N 33/6854
USPC 506/9; 506/18

(57) ABSTRACT

The present invention relates to a method of assessing if a subject is at risk of developing or has already developed asthma, conjunctivitis or rhinitis. The invention further relates to antigen sets for use in such methods including identifying other suitable antigens correlated with asthma, conjunctivitis or rhinitis.

The present invention relates to a method of assessing if a subject is at risk of developing or has already developed asthma, conjunctivitis or rhinitis. The invention further relates to antigen sets for use in the method including methods of identifying antigens correlated with such risk.

### BACKGROUND TO THE INVENTION

Asthma is universally recognized as a major global health challenge. It is one of the most common diseases affecting both adults and children being responsible for up to 300 million cases worldwide and, worryingly, its frequency has increased on a yearly base during the last five decades.

Both genetic (cytokines and immune response genes) and environmental factors such as viral infections, allergens and occupational exposures have been associated with asthma susceptibility, age of onset and severity. However, the pathogenesis of the disease has not been fully elucidated.

A major risk factor is the development of immune responses to foreign antigens that are characterized by the production of antigen-specific IgE. This notion was first inferred from observations showing that the prevalence of asthma was closely related to serum IgE levels. Overwhelming evidence has now confirmed the role of IgE in atopic asthma while several studies have also revealed a link between IgE and non-atopic asthma.

More controversial is the role of antigen specific IgE in determining the onset and severity of the disease. Since the discovery that house dust mites were the major source of allergens in the dust, several studies have linked the presence of serum IgE directed against specific mite allergens and asthma. However, a large number of individuals worldwide particularly those living in some regions of US and Scandinavia are generally not exposed to mite antigens during their life. Interestingly, these individuals do not show any decrease in the prevalence and the severity of asthma. Therefore it is likely that other antigens either alone or in combination play a role in the pathogenesis of the disease.

Unfortunately any link between antigen exposure, IgE production, and occurrence and/or severity of asthma appears to involve an unexpectedly large number of factors and a nonlinear relationship between exposure and response appears to exist.

To date studies attempting to identify a link between specific IgEs and asthma have focused on analysing either one antigen or a few antigens at a time, such as those describing the role of house dust mites. In contrast there are presently no studies that have investigated the IgE response to a large repertoire of antigens and the occurrence of asthma.

The disproportion between the number of known allergens and the number of antigens that have been analyzed may well explain the difficulties encountered in establishing a role for specific IgEs in the pathogenesis of asthma. Most patients with asthma for example, have serum IgE directed to more than one allergen, and the relative contribution of each in the manifestation of the disease and symptoms remains unknown.

Thus, whilst atopy as defined by the presence of specific IgEs to common allergens is associated with asthma, high serum levels of specific IgEs are not always associated with atopy. As a result the association between total or specific IgE and the pattern of asthmatic response remains unclear.

Therefore, while it is relatively straightforward to determine if a patient or subject demonstrates an immune response to a particular antigen, there is no easy way to determine if that same patient or subject is at risk of developing a more severe disorder such as asthma, conjunctivitis or rhinitis. Surprisingly the Inventors have now discovered that it is possible to predict the risk of developing such disorders.

### SUMMARY OF THE INVENTION

In a first aspect of the present invention there is provided a method of identifying a set of biomarkers, more particularly a set of antigens that are significantly associated with asthma, conjunctivitis or rhinitis. The method comprises, (a) measuring in a first plurality of samples isolated from a group of asymptomatic subjects the levels of IgE reactivity to a plurality of antigens, (b) measuring in a second plurality of samples isolated from a group of subjects characterised as having asthma, conjunctivitis or rhinitis the levels of IgE reactivity to the plurality of antigens, (c) identifying a subset of the plurality of antigens which demonstrate significantly different levels of IgE reactivity between the groups of subjects, wherein levels of IgE reactivity to the subset correlate with a clinical diagnosis of asthma, conjunctivitis or rhinitis.

In certain embodiments whilst each individual sample may be assessed separately, the levels of reactivity to the plurality of antigens within each sample are measured at substantially the same time.

Preferably the number of samples in the first and/or second plurality of samples is from 30 to 800 samples, preferably 50 to 800 samples. The number of samples could therefore be any number between 30 and 800; for example 30, 40, 50, 60, 70, 80, 90, 100, 200, 300, 400, 500, 600, 700 or 800, or any range therein. Preferably the plurality of antigens comprises from 30 to 400 antigens, preferably 50 to 400 antigens. The number of antigens could therefore be any number between 30 and 400, for example 30, 40, 50, 60, 70, 80, 90, 100, 200, 300 or 400, or any range therein. In particular embodiments the antigens are selected as being the most prevalent antigens in a specific region.

In one embodiment the levels of IgE reactivity are determined by contacting the plurality of antigens with serum isolated from the groups of subjects and determining the amount of IgE bound to each antigen using an anti-IgE antibody. In particular embodiments the anti-IgE antibody is a labelled antibody. In other embodiments the anti-IgE antibody is unlabelled and is detected using a labelled antibody. The labelled antibody may comprise a fluorescent label. In preferred embodiments the amount of IgE bound to each antigen is determined using fluorescence detection.

In certain embodiments the plurality of antigens are bound to at least one solid support having a plurality of addresses each of which has a distinct antigen disposed thereon. In one embodiment the solid support comprises a plurality of separately identifiable beads. In another embodiment the solid support is a microarray. When the solid support if a microarray the antigens may have a spotting concentration of from 0.008 mg/ml to 3 mg/ml.

In a second aspect the invention provides methods of assessing if a subject is at risk of developing or has developed asthma, conjunctivitis or rhinitis. In certain embodiments the method comprises measuring in a sample isolated from the subject the levels of IgE reactivity to a set of biomarkers. Preferably the biomarkers are antigens pre-determined to correlate with a clinical diagnosis of asthma, conjunctivitis or rhinitis, for example, by way of the first aspect of the invention. In particular embodiments levels of IgE reactivity above 3.51 IU/ml to at least 75% of the set of biomarkers indicates that the subject is likely to develop or has developed asthma, conjunctivitis or rhinitis.

In certain embodiments the set of biomarkers comprises from nine to fifty one antigens. In particular embodiments the set of biomarkers is selected from the group consisting of antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, I6, K87, M1, M3, M4, M5, M6, T4, T6, T7, T9, T14, T901, W1, W6, X902, X903, X904, X905, X907 and X910.

In one embodiment the levels of IgE reactivity are determined by contacting the set of biomarkers with serum isolated from said subject and determining the amount of IgE bound to each antigen using an anti-IgE antibody. In certain embodiments the anti-IgE antibody is a labelled antibody. In other embodiments the anti-IgE antibody is unlabelled and is detected using a labelled antibody. The labelled antibody may comprise a fluorescent label and the amount of IgE bound to each antigen may be determined by fluorescence detection.

In preferred embodiments the antigens are bound to a solid support. In one embodiment the solid support is a microarray. In certain embodiments the antigens have a spotting concentration of from 0.008 mg/ml to 3 mg/ml. Preferably the amount of IgE bound to each antigen in the set of biomarkers is determined at substantially the same time.

In a third aspect, the invention provides an antigen microarray for use in determining the risk of developing or in the diagnosis of asthma, conjunctivitis or rhinitis. The microarray comprises a set of biomarkers correlated with asthma conjunctivitis or rhinitis. In certain embodiments the microarray comprises the antigens F95, G1, G3, G4, G12, G14, G15 and G18 and optionally one or more antigens selected from the group consisting of antigens C2, D1, D2, D3, D70, D71, D72, D73, E1, E3, E81, E82, F4, F16, F25, F35, F49, F84, G2, G5, G6, G8, I6, K87, M1, M3, M4, M5, M6, T4, T6, T7, T9, T14, T901, W1, W6, X902, X903, X904, X905, X907 and X910.

In a fourth aspect of the invention there is provided kits for use in determining the risk of developing asthma, conjunctivitis or rhinitis. In other embodiments the kits may be used in the diagnosis of asthma conjunctivitis or rhinitis. Such kits may comprise, (i) the antigens F95, G1, G3, G4, G12, G14, G15 and G18, (ii) optionally one or more of antigens C2, D1, D2, D3, D70, D71, D72, D73, E1, E3, E81, E82, F4, F16, F25, F35, F49, F84, G2, G5, G6, G8, I6, K87, M1, M3, M4, M5, M6, T4, T6, T7, T9, T14, T901, W1, W6, X902, X903, X904, X905, X907 and X910, (iii) an anti-IgE antibody.

In particular embodiments the kit may comprise the antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, I6, K87, M1, M3, M4, M5, M6, T4, T6, T7, T9, T14, T901, W1, W6, X902, X903, X904, X905, X907 and X910 on a microarray.

In a fifth aspect of the invention there is provided the use of the methods according to the first or second aspects, the microarray of the third aspect or kits according to the fourth aspect for assessing if a subject is at risk of developing or has developed asthma, conjunctivitis or rhinitis.

In a sixth aspect of the invention there is provided the use the methods, microarrays or kits of the previous aspects for screening compounds useful in the treatment of asthma, conjunctivitis or rhinitis.

### BRIEF DESCRIPTION OF FIGURES

FIG. 1: Table 1 lists the 103 antigens that were arrayed and utilised as the primary antigen set for screening. The allergen identifiers correspond with the ImmunoCAP® Allergen product codes, for example, available from Phadia AB.

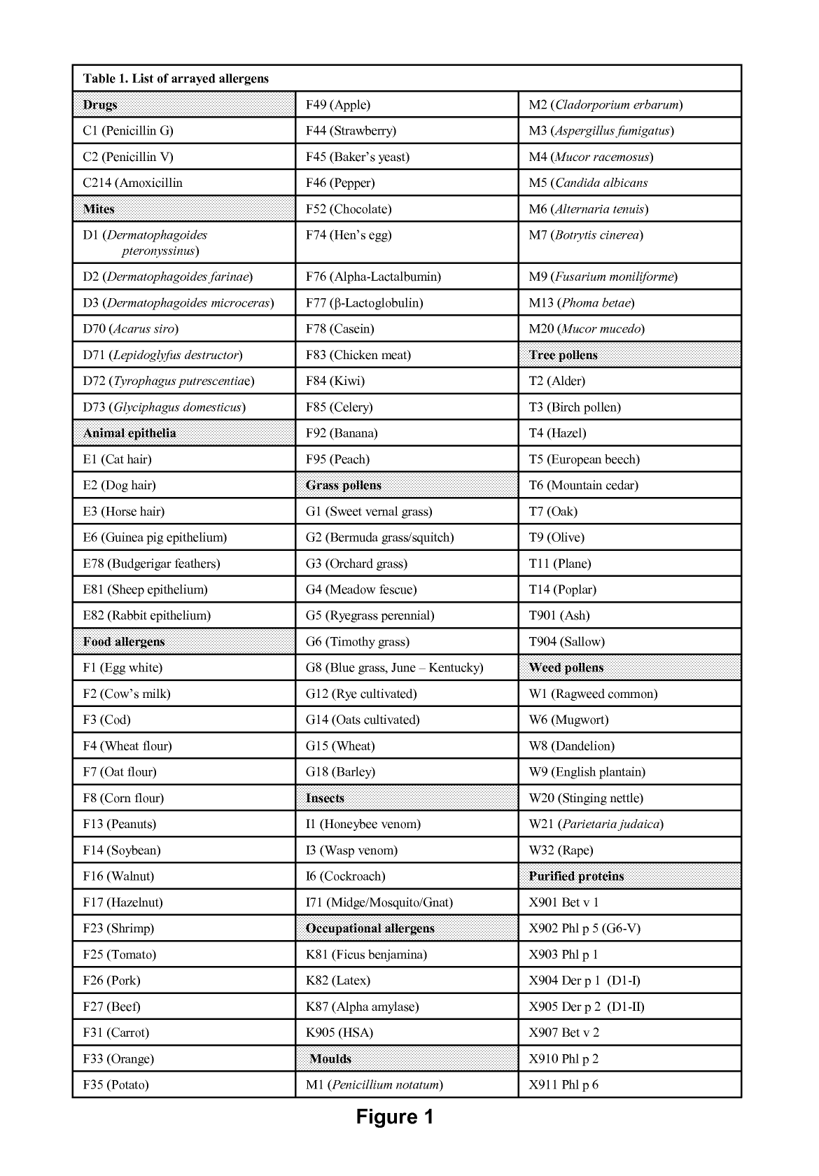

FIG. 2: Table 2 illustrates the stratification of the study population described in the Examples according to age, sex and the occurrence of a number of atopic diseases including asthma.

FIG. 3: Table 3 exemplifies the association analysis of clustered reactivity profiles to the 103 allergens. The three clusters were analyzed for differences in the frequency of sex, conjunctivitis, eczema, rhinitis, asthma as well as asthma—persistency, —severity and age of onset. Associations were assessed using the X² test in SPSS or as in the case of the age of onset by using Kruskal-Wallis rank sum test of equality.

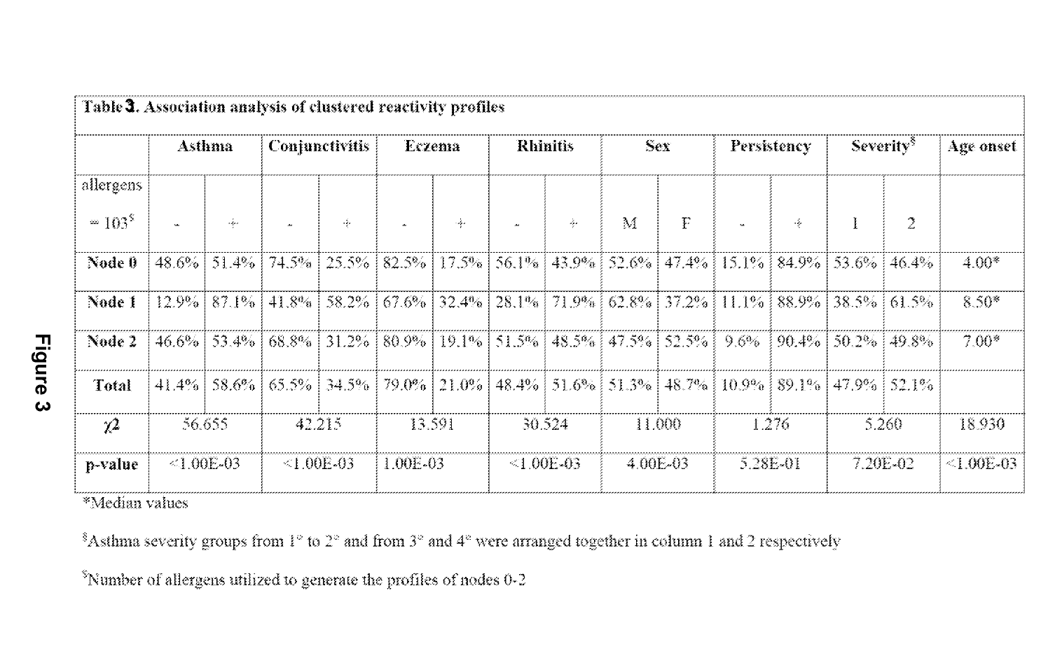

FIG. 4: Table 4 lists the subset of arrayed allergens that showed the highest difference in IgE serum reactivity when comparing asthmatic and non-asthmatic individuals in Mann-Whitney test.

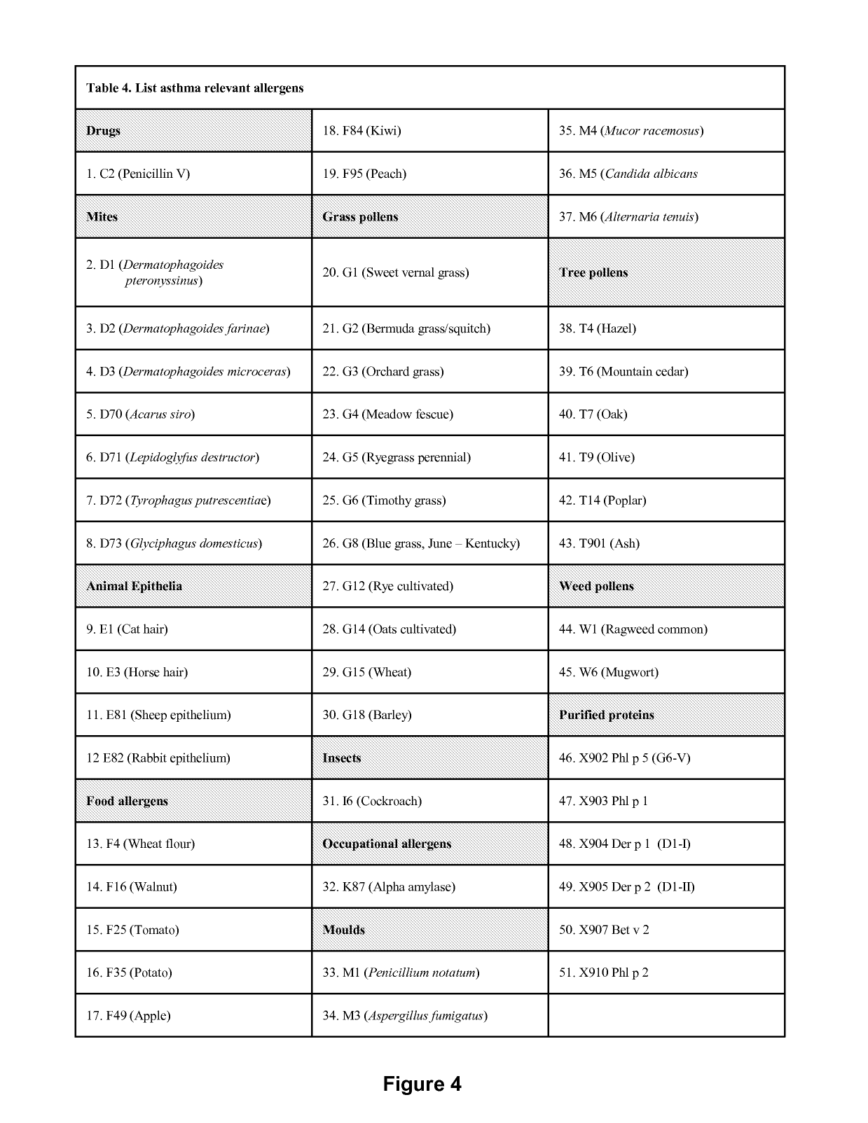

FIG. 5: K-means clustering of serum reactivity profiles of 872 sera (columns) against the arrayed 103 allergens (rows). The specific sera reactivity patterns within each cluster (node) are visualised, FIG. 5a—Node 0; FIG. 5b—Node 1 and FIG. 5c—Node 2. Serum reactivity profiles to individual allergens are classified as either positive (white boxes—class score 1-5) and negative (black boxes—class score 0).

FIG. 6: K-means clustering of serum reactivity profiles of 827 sera (columns) against the arrayed significant selected 51 allergens (rows). The specific sera reactivity patterns within each cluster (node) were visualised, FIG. 6a—Node 3; FIG. 6b—Node 4; FIG. 6c—Node 5. Serum reactivity profiles to individual allergens are classified as either positive (white boxes—class score 1-5) and negative (black boxes—class score 0).

FIG. 7: Associations between cluster and variables distribution were assessed using the Pearson's X2 test, after Bonferroni correction for multiple comparisons. FIG. 7a tabulates the results across all 103 antigens, FIG. 7b tabulates the results across the 51 antigen sub-set.

FIG. 8: Illustrates the generalised architecture and performance of the RBF based ANN asthma classifier. The RBF network consists of three layers: Input (boxes 1-51), hidden (circles 1-8) and output (asthma classes in black boxes) layer respectively.

FIG. 9a: ANN predicted-by-observed performance chart. The box plots represent the predicted-pseudo-probabilities for the RFB output category; asthma (grey) and non-asthmatic (white) plotted against the known clinical status asthmatic (1) asthmatic (2) for combined training and testing samples. FIG. 9b: The ROC curve calculated on the combined training and testing samples, asthma (black), non-asthmatic (grey).

FIG. 10: Illustrates ANN asthma classifier consistency performance The ANN-predicted asthma status of the hold out samples was assessed on a Pearson's X² test against the known clinical status of the selected individuals.

### DETAILED DESCRIPTION OF INVENTION

The inventors have discovered that it is possible to correlate the predisposition or likelihood that a subject will develop or already has developed asthma, conjunctivitis or rhinitis with the presence and/or level of binding of specific IgEs from the subject to particular antigens and antigen sets. The antigens are biomarkers that may be used for prediction, diagnosis or for determining treatment efficacy.

This discovery is surprising because there exist wide variations in the degree of specific IgE binding both between groups of asymptomatic and symptomatic subjects and between individuals within each group. The discovery is a significant advance in the treatment and diagnosis of asthma, conjunctivitis and rhinitis.

The present invention discloses a method for identifying diagnostic biomarkers that have a statistically significant correlation with these clinical conditions. The method identifies differences between asymptomatic and symptomatic patients to reveal previously unrecognised associations between the clinical conditions and the presence or absence of specific IgEs. Thus, disease specific patterns can be generated and used for screening or diagnosis.

To identify disease specific patterns it is first necessary to obtain samples from at least two groups of subjects—one group of healthy, asymptomatic subjects and a second group of symptomatic subjects.

Preferably the symptomatic subjects have been clinically diagnosed with asthma, conjunctivitis, rhinitis or a combination of these. However, it will be apparent to the skilled person that the methods of the invention may also be applied to other, particularly allergic, disorders or diseases such as dermatitis, eczema, inflammation, urticaria, bronchoconstriction and the like.

In order to obtain results or data, generally any sample will normally comprise or be expected to comprise one or more Immunoglobulin E antibodies (IgE) or binding fragments thereof. The sample isolated from a patient or subject is preferably a whole blood sample. Such a blood sample may, for example, be a venous or capillary sample or may be a fraction of such a sample, for example, plasma or serum. Hemolytic, lipemic or icteric samples or samples comprising additives frequently found in vessels used to collect blood, such as EDTA-, heparin- and citrate-, are also envisaged to be suitable for use. Other bodily fluids such as lymph, colostrum, milk, saliva, tears, urine or fractions of such fluids may be utilised. In other embodiments the sample may be derived from a cell culture, cell culture fluid or supernatant or liquefied solid sample, for example from a tissue biopsy.

The patient or subject is a mammal and may be a human or an animal such as, by way of non-limiting example, a cat, dog, rabbit, mouse, guinea pig, ferret, monkey, horse, cow or camel.

In a first step of the method, the levels of IgE reactivity to a plurality of antigens are measured in samples isolated from the subjects in each group.

Subjects may be assigned to a particular group prior to analysis (i.e. symptomatic or asymptomatic) or may be assigned to a particular group following testing, for example, to avoid statistical or scientific bias.

Immunoglobulin E (IgE) directed towards a specific allergen is not normally detected in a sample from a subject and is only produced when a subject becomes sensitised to that allergen. IgE which is directed towards a particular antigen and that will only react with that antigen is generally known as a specific IgE (sIgE). A subject may have specific IgE to more than one allergen.

Reactivity is used to refer to the level of binding between a particular antibody and its ligand to form an immune complex. In the context of the present invention the antibodies are specific IgEs and the ligands are antigens. Generally a specific IgE will bind to a specific antigen. The level of reactivity may be defined in IU/ml or alternatively in terms of ranges which may be assigned Class Score values. For example, Class 0 being less than 0.35 IU/ml; Class 1 being from 0.35 to 0.7 IU/ml; Class 2 being from 0.71 to 3.5 IU/ml; Class 3 being from 3.51 to 17.5 IU/ml; Class 4 being from 17.51 to 50 IU/ml and Class 5 being from 50.01 to 100 IU/ml.

Alternatively, the IgE reactivity to a particular antigen may be considered to be either positive/present or negative/absent, for example below a threshold the response is deemed negative/absent and above the threshold the response is positive/present.

Generally a level of > (greater than) 0.35 IU/mL indicates a positive result, in other words that a specific IgE has bound to its ligand. It is the antibody-antigen complexes formed as a result of interaction between a specific IgE in a sample and its ligand, in this case an antigen, that are measured with a suitable assay.

In the context of this invention, the term allergen means a specific type of antigen that can trigger an allergic response which is mediated by IgE antibody. The method and preparations of this invention extend to a broad class of such allergens and fragments of allergens or haptens acting as allergens. These can include all the specific allergens that can cause an IgE mediated response in allergic subjects. Allergens may be recombinant or prepared from natural sources and may comprise a single epitope or a more complex mixture having two or more epitopes from a single antigen or two or more individual allergens. The terms ‘allergen’ and ‘antigen’ are generally referred to interchangeably.

The term “plurality” as used herein is defined as two, or more than two.

In the context of the first aspect of the invention, the number of samples used should be a number sufficient to produce statistically significant results. For a particular antigen, the results should determine either that there is no correlation between the antigen and specific IgE from a symptomatic subject or that there is a correlation between the antigen and specific IgE from a symptomatic subject; in other words whether or not there is a link between particular antigens and the presence of asthma, conjunctivitis or rhinitis.

Preferably in order to identify any correlation, a large number of individual subjects should be tested, for example, between 30 and 1000 in total, preferably 50 and 1000 in total. Generally only one sample from each individual subject is analysed in each experiment or study.

The number of samples taken from each group may be approximately equal, that is, where 1000 samples are used in total, 500 will come from asymptomatic subjects whilst 500 will come from symptomatic subjects. However, it will be clear to one skilled in the art that the number of subjects and samples may vary depending on a number of factors such as patient recruitment, for example, and will not necessarily be equal. Thus, the number of subjects and samples utilised should be of sufficient number to accurately establish a correlation.

Preferably the plurality of antigens will be a set comprising a large number of antigens, for example greater than 10, 20, 30, 40, 50, 60, 70, 80, 90, 100, more particularly greater than 150, 200, 250, 300, 350, 400, 450 or 500.

The levels of IgE reactivity are determined by contacting each individual sample (comprising specific IgE) separately with a plurality of antigens substantially in parallel and determining the amount of each specific IgE from the sample that is bound to each antigen.

The method may be generally characterised as comprising two separate steps: (a) measuring in a first plurality of samples isolated from a group of asymptomatic subjects the levels of IgE reactivity to a plurality of antigens and (b) measuring in a second plurality of samples isolated from a group of subjects characterised as having asthma, conjunctivitis or rhinitis the levels of IgE reactivity to the plurality of antigens.

For the avoidance of doubt, it will be clear to one skilled in the art that this separation into two separate steps is an artificial separation for the purposes of written description. Essentially the steps are the same, differing only in the group from which the samples are taken. Thus, in the laboratory (a) and (b) may be generalised in terms of a single step repeated for each sample from each group, that is, (ab) measuring in a sample isolated from a subject the levels of IgE reactivity to a plurality of antigens.

For ‘n’ samples, step (ab) would be repeated at least ‘n’ times. When the first plurality of samples comprises ‘x’ samples and the second plurality of samples comprises ‘y’ samples, x+y will therefore equal the number of repetitions, n. It will therefore be apparent that steps (a) and (b) may be carried out in any order, either in parallel or in sequence, or as repeats of step (ab) as necessary until all of the samples are tested.

Preferably the levels of IgE reactivity to a plurality of antigens are measured by means of an immunoassay using an anti-IgE antibody.

Anti-IgE antibodies react with the IgE isotype of human immunoglobulins. Therefore, in certain embodiments the amount of IgE bound to each antigen is determined or quantified using an anti-IgE antibody. The anti-IgE antibody may be a polyclonal, monoclonal, bispecific, humanised or chimeric antibody although affinity-purified antibodies and yet more particularly monoclonal antibodies may generally be preferred. Such anti-IgE antibodies may be conventional or recombinant antibodies and may consist of a single chain but would preferably consist of at least a light chain or a heavy chain. However, it will be appreciated that at least one complementarity determining region (CDR) is required in order to bind a target such as an antigen to which the antibody has binding specificity.

Methods of making anti-IgE antibodies are known in the art. For example, if polyclonal antibodies are desired, then a selected mammal, such as a human, mouse, rabbit, pig, sheep, camel, goat or horse may be immunised with the antigen of choice, such as a heterologous IgE. The serum from the immunised animal is then collected and treated to obtain the antibody, for instance by immunoaffinity chromatography.

Monoclonal anti-IgE antibodies may be produced by methods known in the art. The general methodology for making monoclonal antibodies using hybridoma technology is well known (see, for example, Kohler, G. and Milstein, C, Nature 256: 495-497 (1975); Kozbor et al, Immunology Today 4: 72 (1983); Cole et al, 77-96 in Monoclonal Antibodies and Cancer Therapy, Alan R. Liss, Inc. (1985).

An anti-IgE antibody, as referred to herein, should consist of an epitope-binding region, such as CDR. The antibody may of any suitable class, including IgE, IgM, IgD, IgA and, in particular, IgG. The various subclasses of these antibodies are also envisaged. In particular embodiments, fragments of an anti-IgE antibody or polypeptides derived from such an antibody which retains the binding specificity of the anti-IgE antibody may be used. Such fragments include, but are not limited to antibody fragments, such as Fab, Fab′, F(ab′)2 and Fv, all of which are capable of binding to an epitope.

The term “anti-IgE antibody” also extends to any of the various natural and artificial antibodies and antibody-derived proteins which are available, and their derivatives, e.g. including without limitation polyclonal antibodies, monoclonal antibodies, chimeric antibodies, humanized antibodies, human antibodies, single-domain antibodies, whole antibodies, antibody fragments such as F(ab′)2 and F(ab) fragments, Fv fragments (non-covalent heterodimers), single-chain antibodies such as single chain Fv molecules (scFv), minibodies, oligobodies, dimeric or trimeric antibody fragments or constructs, etc. The term “anti-IgE antibody” does not imply any particular origin, and includes antibodies obtained through non-conventional processes, such as phage display. Antibodies of the invention can be of any isotype (e.g. IgA, IgG, IgM i.e. an α, γ or μ heavy chain) and may have a κ (kappa) or a λ (lambda) light chain.

The invention therefore extends to the use of anti-IgE antibodies and binding fragments which have binding specificity to IgE for use in the present invention.

The term “specifically binds” or “binding specificity” refers to the ability of an antibody or fragment thereof to bind to a target with a greater affinity than it binds to a non-target epitope. For example, the binding of an antibody to a target epitope may result in a binding affinity which is at least 10, 50, 100, 250, 500, or 1000 times greater than the binding affinity for a non-target epitope. In certain embodiments, binding affinity is determined by an affinity ELISA assay. In alternative embodiments, affinity is determined by a BIAcore assay. Alternatively, binding affinity may be determined by a kinetic method.

In particular embodiments the anti-IgE antibody is a labelled antibody. By way of non-limiting example labelling may be by conjugation to an enzyme such as a peroxidise or a chemiluminescent or fluorescent compound, such as Alexa Fluor 555 or a mass tag.

In other embodiments the anti-IgE antibody is unlabelled and is detected using a further antibody, commonly called a secondary antibody, which may be labelled as described.

In a preferred embodiment, the anti-IgE antibody is an unlabelled mouse monoclonal antibody directed against IgE and which is detected using a labelled secondary antibody, such as an anti-mouse IgG. In certain embodiments, one or more luminescent or fluorescent moieties may be bound to avidin/streptavidin, which in turn may be bound to biotin chemically conjugated to an antibody. In certain further embodiments, lectins (Protein A/G/L) can be linked to a luminescent or fluorescent molecule which may also be attached to an antibody or other protein conjugate. In preferred embodiments a tyramide signal amplification system is utilised that uses the catalytic activity of horseradish peroxidase (HRP) to generate high-density labelling of an antibody. Suitable labels and labelling methods are known in the art and would be apparent to the skilled artisan.

In particular embodiments the labelled antibody comprises a fluorescent label.

Appropriate fluorescent labels are well known in the art, and can include, by way of non-limiting example, Alexa Fluor 488, Alexa Fluor 555, R-phycoerythrin, Aqua, Texas-Red, FITC, rhodamine, a rhodamine derivative, fluorescein, a fluorescein derivative, cascade blue, Cy5 or Cy3.

The detection method may be by any suitable method known in the art such as by optical detection including fluorescence measurement, colourimetry, flow cytometry, chemiluminescence and the like. Other methods include electrochemical, radioactive, piezoelectric methods and the like. Yet other methods of detection of binding may be by surface Plasmon resonance (SPR), surface Plasmon microscopy (SPM), surface Plasmon fluorescence spectroscopy or SELDI mass spectroscopy and the like. The skilled person will appreciate that detection may be performed using a combination of detection methods.

Particularly, detection of binding is by measurement/detection of a luminescent signal, for example, chemiluminescent light produced by a chemiluminescent compound.

Whilst many of the antigens that a population is exposed to will be common between one geographical region and another, there may be particular antigens that are common in some regions but rare in others. Therefore, for each analysis or each time the method is performed, suitable antigens may be selected from the most prevalent antigens in a specific geographical region or country and may vary. A suitable plurality of antigens, specifically 103 antigens, is identified in table 1. One skilled in the art will be able to select appropriate antigens and suitable antigens and antigen preparations are widely commercially available.

Whilst each individual sample will generally be assessed separately, the levels of reactivity to the plurality of antigens within each sample are preferably measured at substantially the same time. By way of example, if the plurality of antigens comprises 50 antigens, for a group of 1000 subjects, 1000 separate assays will be carried out and the results of 50,000 reactions measured. Measuring such a large number of reactions one by one, whilst possible, is generally not feasible and would take a significant amount of time and persons to complete.

Thus, to facilitate parallel processing of large numbers of antigens, preferably the plurality of antigens are bound to at least one solid support having a plurality of addresses each of which has a distinct, i.e. specific, antigen disposed thereon. The use of a solid support is advantageous since it enables parallel processing of a large number of antigens whilst minimising the time and effort required.

The solid support may be any material known in the art, for example, a glass carrier, synthetic carrier, silicon wafer or membrane. Suitable materials include plastics, glasses, silicon, ceramics or organic polymers including polystyrene, polycarbonate, polypropylene, polyethylene, cellulose and nitrocellulose. The surface itself may be in the form, or part, of a slide, sheet, microplate or microtitre plate, tray, membrane, fibre, well, pellet, rod, stick, tube, bead and the like.

Use of the term “bound” is intended to mean that a substance, in this case an antigen, is retained, immobilised or substantially attached to a surface at the molecular level (i.e., through a covalent or non-covalent bond or interaction). The immobilisation method used should be reproducible, applicable to antigens of different properties (size, hydrophilic, hydrophobic), amenable to high throughput and automation, and maintain the ability of the antigen to be form a complex with an antibody. By way of non-limiting example, suitable methods known in the art include passive adsorption, affinity based binding, covalent coupling to chemically activated surfaces, photochemical cross-coupling and the like.

The term “address” is used to refer to a distinct feature of a solid support or defined location on a solid support that allows a specific antigen to be identified enabling the level of IgE reactivity to that specific antigen to be determined.

In one embodiment the solid support is a plurality of beads each being separately identifiable by means of an address. Suitable addresses include RFID tags, mass tags, fluorescent tags, optical encoding, digital magnetic tags, spectrometric encoding and the like.

In other embodiments the solid support is a microarray. The term “microarray” as used herein, refers to an ordered array of spots presented for binding of IgE antibodies. Microarrays of the present invention comprise at least two, at least nine, at least fifty, at least one hundred, at least five hundred or at least one thousand spots. In some embodiments the microarray may comprise at least 10,000, 40,000, 100,000, or 1,000,000 different and distinct spots. The spots may be at a density of from about 100/cm² to about 1000/cm² or greater.

A “spot” refers to a reagent or reagents, in this instance a specific antigen or allergen preparation, deposited at a particular address, in this instance a physical location, on the array surface. Typically a spot is characterised by the presence of one or more specific molecules (e.g. particular proteins, allergen extracts, antigens, etc.). Spots may be from 10 to 2000 μm in diameter, 50-500 μm in diameter or 150-250 μm in diameter. Antigens may be applied to the surface of a solid support at a spotting concentration of from 0.008 mg/ml to 3 mg/ml in order to form an array.

Systems suitable for measuring or reading fluorescence signals from microarrays are known. Generally an image is constructed by scanning the slide in two dimensions under a laser spot. An image can be acquired in about one minute, but the analysis is complicated in terms of the image analysis processes. These processes can be complex because of both the large amount of data generated and the analysis algorithms required to produce an unambiguous measurement of the integrated signal from each spot or micro-spot.

In their previous patent application published as WO/2003/091712, the inventors realised that it is possible to read the fluorescence by illuminating the entirety of each spot on an array and taking a measurement of the fluorescence of the entire spot in a single measurement rather than scanning across and illuminating a fraction of each spot several times in order to build up an image of each spot pixel by pixel. This approach enables LEDs, which are low cost light sources, to be used as the illumination source. Each fluorescent molecule receives the same optical energy as it would do if a coherent light source was used as the illumination source. However, the detector yields a single reading requiring no further signal analysis (rather than a 400 pixel image per spot which requires complicated image processing to calculate an overall measurement). Use of a coherent light source may in some circumstances be a disadvantage, because of additional noise introduced in the signal arising from the interference effects.

In other embodiments the use of microcantilevers is envisaged as disclosed in International Patent publication WO2006/138161. In yet other embodiments the assay is carried out using one or more microfluidic chips.

A third step of the method comprises, (c) identifying a subset of the plurality of antigens which demonstrate significantly different levels of IgE reactivity between the groups of subjects, wherein levels of IgE reactivity to the subset correlate with a clinical diagnosis of asthma, conjunctivitis or rhinitis.

Significantly different levels of IgE reactivity between the groups of subjects may be determined using statistical data analysis methods. Statistical analysis of the data set may be used to determine if one or more antigens is/are connected, i.e. correlated, with a clinical diagnosis of asthma, conjunctivitis or rhinitis.

For example, subjects may be divided into at least two classes based on the IgE reactivity levels to specific antigens. The correlation between IgE reactivity and health status is then analysed by a cluster or learning algorithm. By way of non-limiting example, suitable methods include supervised and/or unsupervised clustering algorithms, k-means, principal component analysis, hierarchical clustering, nearest neighbour analysis, support vector machines, artificial neural networks and the like. A substantial number, for example at least 50%, 60%, 70%, 80%, 90% or greater) of subjects in a particular cluster or class may have a first health status and a substantial number of patients in one or more other classes may have a different health status.

Differentials in the reactivity of IgE to the plurality of antigens in a first group of subjects relative to another group of subjects can thus be identified. The level of reactivity to such antigens may be used as biomarkers for predicting/determining health status, clinical outcome or treatment outcome in a subject. The reactivity levels identified and which are correlated with a clinical condition represent an idealised pattern of reactivity that is representative of subjects of different health status. As a result such disease specific patterns or profiles may be compared to predict/determine health status, clinical or treatment outcome in a subject.

Suitable methods are exemplified below and include cluster analysis to assign the data into subsets or clusters.

The end point of the analysis is the identification of a subset of antigens from the plurality of antigens which are correlated or linked with a clinical diagnosis of asthma, conjunctivitis or rhinitis. The IgE reactivity to the subset of antigens may be used to prepare a generalised profile of reactivity for a particular clinical condition that may be used for comparison. The identification of a subset of 51 antigens correlated with such a clinical diagnosis is exemplified in the experimental section below. The antigens comprising the antigen subset are listed in table 4. It will be apparent that the composition of any particular subset will depend on the composition of the plurality of antigens that are used. It will be further apparent that subsets of antigens linked with a clinical diagnosis may be combined. For example, if a first set comprises 20 antigens and a second subset comprises 30 antigens, assuming that there are no antigens in common between the subsets, a combined subset will comprise 50 antigens. However, it is likely that there will be some commonality between subsets such that the total number of antigens in a combined subset may be less than the sum of the number of antigens in each subset prior to combination. In this context, use of the term combination simply means that the antigens are used together, for example positioned separately on the same microarray.

The second aspect of the invention provides a method of assessing if a subject is at risk of developing or has developed asthma, conjunctivitis or rhinitis.

The method comprises the step of (a) measuring in a sample isolated from said subject the levels of IgE reactivity to a set of biomarkers.

In the context of the present invention at least a proportion of the biomarkers will be derived from a subset of antigens correlated with a clinical diagnosis of asthma, conjunctivitis or rhinitis. Preferably the antigens have been identified according to the first aspect of the invention. Thus, such antigens are already known to correlate with a clinical diagnosis. Hence, the term “pre-determined to correlate” as used herein refers to an element, for example an antigen, the reactivity to which has demonstrated a statistically significant link with a disease or disorder of interest prior to its use in the method. Suitable methods for determining such a correlation are exemplified in the experimental section below and according to the first aspect of the invention.

By use of the term proportion is meant that at least 10% of the biomarkers are antigens pre-determined to correlate with a disease or disorder of interest. More particularly at least 15%, 20%, 25%, 30%, 35%, 40%, 45% or 50% alternatively at least 55%, 60%, 65%, 70%, 75%, 80%, 85%, 90% or 95% of the biomarkers are pre-determined to correlate with a disease or disorder of interest. In certain embodiments all of the biomarkers (100%) are pre-determined to correlate with a disease or disorder of interest.

Similarity or difference between the IgE reactivity profile of a subject is indicative of the class membership of the subject and their respective health status. Similarity or difference may be determined by any suitable means, for example by statistical methods outlined above. The comparison may be qualitative, quantitative or both.

The IgE reactivity to the subset of antigens may be used to prepare a profile of reactivity that may be used for comparison with a generalised profile for a particular clinical condition. Comparison of the biomarker profile of a subject may be compared with a reference profile, either manually or electronically. In one example the comparison is performed by comparing each IgE reactivity level to a specific antigen with a generalised IgE reactivity level to a specific antigen in a reference data set or profile. The IgE reactivity to a specific antigen may have an absolute or normalised value. The difference between the IgE reactivity to a specific antigen of a subject and a reference profile or data set may be assessed by fold changes, absolute differences, pattern recognition or comparison or other suitable means including algorithms.

In certain embodiments in a sample from a subject, levels of IgE reactivity above 3.51 IU/ml to at least 75% of the set of biomarkers indicates that the subject is likely to develop or has developed asthma, conjunctivitis or rhinitis.

In other embodiments the levels of reactivity may be above 0.71 IU/ml above 3.51 IU/ml, above 17.51 IU/ml or above 50.01 IU/ml. It will be apparent that there will be variances in IgE reactivity and response to antigens. Therefore, preferably a positive reaction to at least 75%, at least 80%, at least 85%, at least 90% or at least 91%, 92%, 93%, 94%, 95%, 96%, 97%, 98%, 99% or 100% of the plurality of antigens indicates that the subject is likely to develop or has developed asthma, conjunctivitis or rhinitis.

It will also be apparent that there may be particular threshold levels of reactivity that are indicative of a clinical condition and but which differ between antigens. Hence for the purposes of diagnosis or prediction a reactivity level of 17.51 IU/ml may indicate a positive result for one antigen but a negative result for another.

In particular embodiments the set of biomarkers will comprise from 9 to 51 antigens pre-determined to correlate with a disease or disorder or interest.

Particular antigens pre-determined to correlate with a disease or disorder of interest include antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, 16, K87, M1, M3, M4, M5, M6, T4, T6, T7, T9, T14, T901, W1, W6, X902, X903, X904, X905, X907 and X910. Identification of these antigens is exemplified in the experimental section below. In particular embodiments at least antigens F95, G1, G3, G4, G12, G14, G15 and G18 are preferably included.

As discussed above in relation to the first aspect of the invention, the levels of IgE reactivity may be determined by contacting the plurality of antigens or biomarkers with serum isolated from a subject and determining the amount of IgE bound to each antigen using an anti-IgE antibody.

The detection of IgE antibodies indicates that the sensitisation process has been initiated. Along with symptoms and a positive case history it confirms a clinical diagnosis alternatively without symptoms it may predict later development of allergic disease. It is often seen that specific IgE antibody responses precede the symptoms, but the symptoms develop later over time.

Again, and as stated with regard to the first aspect of the invention the antigens are bound to a solid support.

Thus, in a third aspect of the invention there is provided an antigen microarray for use in a method for assessing if a subject is at risk of developing or has developed asthma, conjunctivitis or rhinitis which comprises the antigens F95, G1, G3, G4, G12, G14, G15 and G18 and optionally one or more antigens selected from the group consisting of antigens C2, D1, D2, D3, D70, D71, D72, D73, E1, E3, E81, E82, F4, F16, F25, F35, F49, F84, G2, G5, G6, G8, I6, K87, M1, M3, M4, M5, M6, T4, T6, T7, T9, T14, T901, W1, W6, X902, X903, X904, X905, X907 and X910. Antigen microarrays that are not used according to the first or second aspects of the invention or that are not for use in assessing if a subject is at risk of developing or has developed asthma, conjunctivitis or rhinitis are preferably explicitly disclaimed.

In a fourth aspect of the invention there are provided kits comprising (i) the antigens F95, G1, G3, G4, G12, G14, G15 and G18 and (ii) optionally one or more of antigens C2, D1, D2, D3, D70, D71, D72, D73, E1, E3, E81, E82, F4, F16, F25, F35, F49, F84, G2, G5, G6, G8, I6, K87, M1, M3, M4, M5, M6, T4, T6, T7, T9, T14, T901, W1, W6, X902, X903, X904, X905, X907 and X910. The antigens may be provided in a form suitable for use in the methods according to the first and second aspects or in the preparation of a micro array according to the third aspect of the invention.

Such kits may include further components, for example, an anti-IgE antibody, wash buffers, diluents, antibodies (i.e. primary, secondary, tertiary), detection reagents, fluorophores, gloves, pipette tips, instruction manuals and the like. Preferably the antigens are provided in the form of spots on a microarray and may also include controls and standards, etc.

Thus the kit may comprise an array consisting of antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, 16, K87, M1, M3, M4, M5, M6, T4, T6, T7, T9, T14, T901, W1, W6, X902, X903, X904, X905, X907 and X910. Alternatively the kit may comprise an array comprising antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, 16, K87, M1, M3, M4, M5, M6, T4, T6, T7, T9, T14, T901, W1, W6, X902, X903, X904, X905, X907 and X910.

In a fifth aspect of the invention, and as exemplified in the experimental section, there is provided use of the method of the first and second aspects, the microarray of the third aspect or the kit of the fourth aspect for assessing if a subject is at risk of developing or has developed asthma, conjunctivitis or rhinitis.

In a sixth aspect of the invention there is provided the use of the methods, microarrays or kits of the previous aspects for screening compounds useful in the treatment of asthma, conjunctivitis or rhinitis.

For example, the method may comprise the steps of: (a) measuring in a first sample isolated from a subject the levels of IgE reactivity to a set of biomarkers; (b) measuring in a second sample isolated from the subject the levels of reactivity to a set of biomarkers and (c) comparing the levels of IgE reactivity to determine any differences characterised in that a proportion of the biomarkers are antigens pre-determined to correlate with a clinical diagnosis of asthma, conjunctivitis or rhinitis.

In one embodiment the first sample maybe taken from a subject prior to treatment with a pharmaceutical or drug. The second sample may be taken from the same subject following treatment with a pharmaceutical or drug. Any changes in the levels of reactivity between the first and second samples may be indicative that a particular pharmaceutical or drug is of use or has clinical application in the treatment of asthma, conjunctivitis or rhinitis. Alternatively such results may indicate the efficacy of a treatment regime for an individual subject.

It should be apparent that between or at each stage of the methods according to the first, second, fifth and sixth aspects, optional washing, drying and/or incubation steps may be included. The methods may also optionally include ‘blocking steps’ between one or more steps of the methods, for example wherein a concentrated solution of a non-interacting protein, such as bovine serum albumin (BSA) or casein, is added, for example to all wells of a microtitre plate. Such proteins block non-specific adsorption of other proteins and may be beneficial in reducing ‘background’ artifacts which can interfere with the sensitivity of an assay.

The inventions may be better appreciated by reference to the following description and examples which are intended to be illustrative of the methods of the invention.

### EXAMPLES

### Population Case Study

Families with progeny suffering from atopic asthma were included in this study. All asthmatic patients were atopic. Atopic asthmatic sibling pairs (sibs) and trios were collected over a period of 4 years, mainly from pediatric and pneumologic centers. To avoid phenocopies, all patients fulfilled the following criteria: Sardinian origin for at least 3 generations and age at visit >6 years.

At the recruitment sessions, each subject was interviewed, disease status was ascertained by physical examination, permission was asked to access personal health records, and blood samples were collected. Each participant signed an informed consent form approved by the local ethics committee (Azienda Sanitaria Locale number 8 protocol 24/Comitato Etico/02, authorization number 4737). Asthma was diagnosed by a pulmonary physician, in accordance with American Thoracic Society criteria (1) Pulmonary function was evaluated by spirometry: forced expiratory volume at the 1st s (FEV1) was expressed in liters/minute. A physician administered a questionnaire collecting clinical history and classifying asthma severity in four levels according to the World Health Organization guidelines (Global Initiative for Asthma). The use of asthma drugs and any other medication was recorded. Atopy was detected by positive skin testing to common inhalant allergens by standard methods. Patients with history of early onset were interviewed by a physician about persistency of asthma symptoms after the completion of puberty (18 years).

The sample consisted of a total of 872 sera, including 440 parents and their progeny (432 individuals). Within the study group, 428 children and 58 parents (55.73% of the total) were diagnosed with asthma, 341 parents (39.11% of the total) were classified as non asthmatic though some of them suffered from atopy related disorders such as rhinitis, conjunctivitis and eczema, a remaining 5.16% were classified as unconfirmed asthma diagnosis. These data are tabulated in Table 2.

### Development of a Microarray-Based Immunoassay

The microarray immunoassay procedure consisted of four phases (printing', processing, scanning, quantification and analysis).

### Preparation of Microarray

To generate the array selected allergens (natural extracts, purified allergens and recombinant molecules) were printed onto aldehyde-activated glass microscope slides in duplicates at scrambled positions to minimize the effect of processing errors. The array also included positive and negative controls, blanks and an internal dose response curve.

To reduce the standard spotting time, decrease the inter-batch variability and increase the number of chip processed per test, 2 chips (“allergochip”) were printed onto a single microscope slide. Each microarray batch included 120 slides, with a total number of 240 allergochips (the execution time was 14 hours).

The analysis of the study group required a total of 480 printed slides, corresponding to 960 chips and the overall printing procedure was divided into four batches. Each chip contained 103 allergens printed in duplicate onto aldehyde-activated glass microscope slides (CEL Associates) using high—speed robotics (Microgrid Compact; Biorobotics).

Arrays were ‘printed’ at 23° C./60° C. humidity and stored overnight inside the printing cabinet. Allergens (provided by Allergopharma) were initially reconstituted in PBS pH7.4 (reconstitution buffer) with a final concentration ranging form 0.4 to 40 mg/ml and after that spotted onto the arrays in the following spotting buffer PBS pH7.4, glycine pH2.4, Borate pH9.4, glycerol 10%, DTT 5 mM, SDS (0.2%; 0.05%), Tween 20 (0.01%). The spotting concentration for each allergen ranged from 0.008 to 3 mg/ml.

### Microarray Processing

Printed slides were blocked with PBS containing 2% BSA for 1 h at room temperature. Slides were then incubated with individual serum samples (100 μl) for 60 minutes at 37° C. To reveal bound IgE, the slides were incubated with a secondary mouse monoclonal antibody directed against human IgE (0.14 μg/ml-100 μl) for 45 minutes at 37° C., followed by an incubation with anti-mouse IgG HRP conjugated antibody (1.6 μg/mL-100 μl) for 45 minutes at 37° C. and finally incubated with tyramide-Alexa 555 (TSA™ Kit #42 *with HRP-streptavidin and Alexa Fluor® 555 tyramide* *50-150 slides*) (Invitrogen) diluted 1:200 (100 μl), for 15 minutes at 37° C. Slides were dried at 37° C. before measuring the fluorescence signal.

### Fluorescence Measurement

The processed slides were scanned using a fluorescence-detecting scanner ScanArray™ Gx and the images were generated with the ScanArray™ software provided by Perkin Elmer Life Sciences Inc. All the slides were scanned under identical settings: 90% laser power and 60% photomultiplier gain.

Quantification of Bound IgE

The fluorescence signal was acquired using ProScanArray Express™ version 3.0 software. PMC reading values of individual spots were corrected against the internal negative control to identify signals above background. Duplicate measurements of individual allergens were utilized.

Concentrations (IU/ml) of allergen-bound IgE were determined by interpolation with an internal calibration curve printed onto each microarray. The calibration curve consisted of decreasing amounts of streptavidin (80; 53.3; 35.6; 23.7; 15.8; 10.5; 7.02 μg/ml) that capture myeloma biotinylated IgE spiked into the blocking solution. To assign IU/ml values to the calibration curve, the average signals collected at different amounts of printed streptavidin were interpolated with an external Reference Curve generated by microarray slides printed with replicates of Goat anti-Human IgE and incubated with increasing concentrations of human IgE (WHO Reference standard 0.35, 1.0, 3.5, 10.0, 50.0 IU/ml).

The signal collected from the allergens was interpolated with the calibration curve to obtain the IU/ml value, and translated into a Class Score by plotting the data in a standard reactivity scale. Class Score values: (CLASS 0 (less than 0.35 IU/ml); CLASS 1 (0.35-0.7 IU/ml); CLASS 2 (0.71-3.5 IU/ml); CLASS 3 (3.51-17.5 IU/ml); CLASS 4 (17.51-50 IU/ml); CLASS 5 (50.01-100 IU/ml).

The serum IgE reactivity was analyzed using a fluorescence immunoassay that incorporates as a substratum a microarray of 103 allergens representative of 11 distinct allergen classes chosen amongst those most frequently associated with atopic diseases in Southern-Central Europe.

### Analysis of Serum Reactivity Profiles

The reactivity profiles generated by incubating the sera with the array immunoassay were analyzed using k-means clustering after encoding each serum reactivity profile with 103-dimensional vectors (1 dimension for each allergen) using Cluster 3.035 software while MapleTree was used for visualizing the clustering results.

Profile partitioning attempts were made using different numbers of clusters to identify the condition that maximize intra cluster similarities and inter cluster differences. For this purpose we analyzed the clustered profiles with several performance indicators, as provided by the Machaon37, 38 software, to validate the statistical significance of each partitioning attempt. Under the condition k=3 (i.e. k-means clustering configured to obtain three clusters) four out the five performance indicators, as computed by Machaon scored the highest values.

The clusters generated using k-means clustering at k=3 were analyzed to search for associations with age, sex, and the presence of a pathological condition (asthma, rhinitis, etc.), its persistency and/or severity, onset age, etc. Statistical tests such as Pearson's) X2 and Kruskall-Wallis non-parametric test were run within the SPSS software and Excel to investigate whether the frequency of a pathological condition differed significantly in the clusters and whether each cluster significantly differed from the study population taken as a whole.

### Clustering Analysis

We used k-means clustering to group sera into distinct clusters on the basis of similarities in their reactivity profiles. The k-means algorithm is an unsupervised, iterative algorithm that partitions objects into a fixed, user-defined number (k) of clusters, such that the clusters are internally similar but externally dissimilar. To define distances between sera, the Euclidean distance similarity metric was used.

Several iterations were carried out to minimize the sum of distances within each cluster and to provide the optimal clustering solution by convergence. This analysis showed that convergence to an optimal solution was achieved within 10000 iterations of the algorithm.

A number of statistical tests were utilised having different k values (from 1 to 14 and 20) to identify the partitions that generated clusters that significantly differed from each other. They included:

(1) The Silhouette Validation method defines the concept of silhouette as an indicator of cluster tightness and separation. A good partitioning process results in clusters that are tight (i.e. objects within each cluster are close to each other) and well separated (i.e. clusters are far from each other), while a bad partitioning generates clusters much closer to each other (to the point where they may be partially overlapping) and objects within clusters may be more scattered, thus making it difficult to assign some samples to a specific cluster. The mathematical model of the silhouette for the i-th object, S(i), is:

$$S ( i ) = \frac{( b ( i ) - a ( i ) )}{max \{ a ( i ) , b ( i ) \}}$$

where a(i) is the average dissimilarity of i-th object to all other objects in the same cluster; b(i) is the minimum of average dissimilarities of i-th object to all objects in the other, closest, cluster. S(i) ranges from −1 (misclassified object) to 1 (optimal partitioning result for the object). The arithmetic average of the S(i) for all the clustered objects provides the overall performance of the entire clustering process. The closer the index to 1, the better the clustering result.

(2) Dunn's Validity Index. Similar to the Silhouette method, this technique is also based on the concept that a good partitioning generates compact and well-separated clusters. The index that models such attributes, the Dunn's validation index D, is defined as follows:

$$D = \text{?} \{ \text{?} \{ \frac{d ( {c}_{i} , {c}_{j} )}{\text{?} \{ {d}^{′} ( {c}_{k} ) \}} \} \} , \\ \text{?} \text{indicates text missing or illegible when filed}$$

where d(ci,cj) is the distance between clusters ci, and cj (inter cluster distance); d′(ck) is the intra cluster distance of cluster ck, n is the number of clusters. In a good partitioning process, the inter cluster distances are maximized (max. separation between clusters) while the intra cluster distances are maximized (max compactness within cluster). The higher D, the better the clustering result.

(3) Davies-Bouldin Validity Index. The same concepts of tightness (compactness) and separation are captured also by the DB index, which is defined as follows:

$$DB = \frac{1}{n} {\sum }_{i = 1}^{n} {max}_{i \neq j} \{ \frac{\text{?} ( {Q}_{i} ) + \text{?} ( {Q}_{j} )}{S ( {Q}_{i} , {Q}_{j} )} \} , \\ \text{?} \text{indicates text missing or illegible when filed}$$

where n is the number of clusters, sₙ is the average distance of all objects belonging to the cluster to their cluster centre (measure of compactness) and s¹Q₁Q₂) is the distance between cluster centers (measure of separation). DB is small if the clusters are compact and far from each other (good clustering result).

(4) C index. The C index is defined as follows:

$$C = \frac{S - {S}_{m i n}}{{S}_{ma x} - {S}_{m i n}}$$

Considering a single cluster, and assume that all of its objects are organized into pairs. Say there are I pairs in a given cluster. Then S is the sum of distances of those I pairs. This can be seen as a measure of compactness of the cluster. Considering the entire set of objects (i.e. all the clusters), another sum of distances 5 min is computed by taking the I smallest distances that can be found amongst all possible pairs (i.e. both within and between clusters). Similarly, Smax is computed as the sum of the I largest distances out of all pairs. Given such definitions, a small value of C is an indicator of a good clustering result.

(5) Isolation index. This technique is based on the assumption that if an object belongs to a cluster, its nearest neighbours are likely to belong to the same cluster as well. This property should be maximized in a good clustering result. To capture this concept mathematically, the following expression is introduced:

$${l}_{k} = \frac{1}{n} \text{?} {v}_{k} ( {x}_{i} )$$

$$\text{?} \text{indicates text missing or illegible when filed}$$

where xi is the i-th object, vk(xi) is the fraction of nearest neighbours of xi that have been correctly assigned to the same cluster, and n is the total number of objects in the dataset. For a good clustering result, a high value of Ik is desired for each object in each cluster.

For each individual we obtained an IgE reactivity profile against 103 distinct allergens chosen amongst those most commonly associated with atopic diseases in Southern-Central Europe. The analysis of the individual profiles showed a remarkable level of diversity in the IgE recognition pattern in terms of class scores and combinations of allergens recognized. Very few profiles were identical and a number of otherwise healthy individuals showed a surprisingly complex pattern of reactivity against a number of allergens. Such diversity likely reflects differences in allergen exposure and the genetic diversity of the study populations.

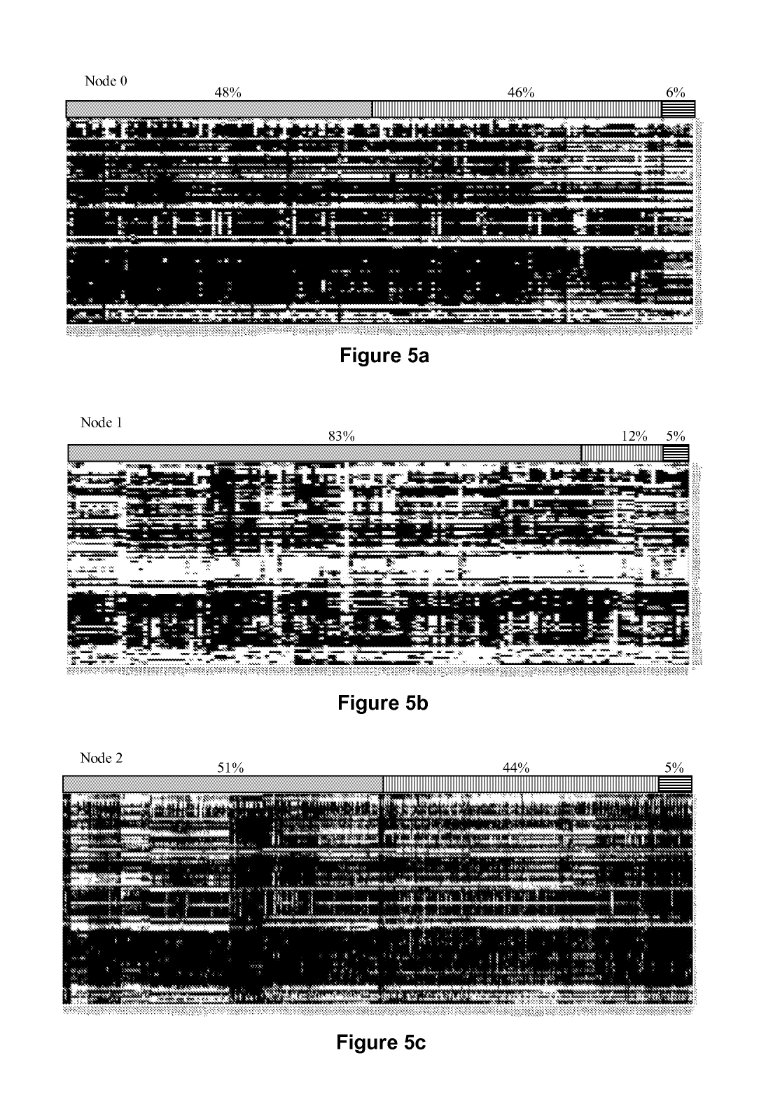

To identify groups showing similarities in the IgE serum reactivity we processed the profiles using k-means clustering at setting that we experimentally validated to maximize inter cluster similarities and intra cluster differences. The IgE reactivity profiles for each cluster, or node are shown in FIGS. 5a, 5b and 5c. This analysis generated three clusters that significantly differed form each other in term of the combination of allergens recognized and in the proportion of individuals affected by different atopic diseases including asthma, rhinitis and conjunctivitis. In particular asthmatic individuals contributed to 83% of the reactivity profiles of cluster 1. This percentage showed a remarkable statistical significant difference (p<1E-8) compared to that of the asthmatic individuals in the study population (Table 3).

Interestingly while cluster 0 and cluster 2 did not show significant differences in the proportion of asthmatic and non-asthmatic individuals the analysis of their composition demonstrated that, contrary to cluster 1, they were enriched in members of the same family nuclei. We thought that the composition of cluster 0 and 2 reflected the exposure of family members to a common set of allergens that were included in the microarray. These allergens though having a powerful sensitizing ability were not relevant for asthma but contributed to the formation of the reactivity profiles and therefore could mask additional associations.

### Identification of Asthma Relevant Allergens

Asthmatic and non-asthmatic individuals were compared for the reactivity against each allergen using the Mann-Whitney test to identify those allergens that showed the most significant differences in the two groups.

The study sample fulfilled the requirement to utilize the Mann-Whitney test: i) the asthmatic and non-asthmatic group were regarded as independent, ii) the sera reactivity values can be regarded as an ordinal random variable; iii) the number of cases to be compared is sufficient. Furthermore this test does not require a specific distribution of values (e.g. Gaussian) and can be performed using two groups with different numbers of individuals (although similarity gives better performance).

### Data Analysis

The reactivity data for each allergen was flagged in the input file depending on the asthmatic status of the referring individuals. Asthmatic (corresponding to a flag named “2”), not asthmatic (corresponding to a flag named “1”). Sera corresponding to individuals with an unconfirmed asthmatic clinical status (5.16%) were manually depleted from the file. The total amount of sera used for this analysis after eliminating individuals with unconfirmed diagnosis was 827. The starting number of allergens present was 103. The reactivity values for each allergen ranged from 0 to 5 (microarray recorded class score). Using SPSS, the data was converted to ordinal before performing the analysis.

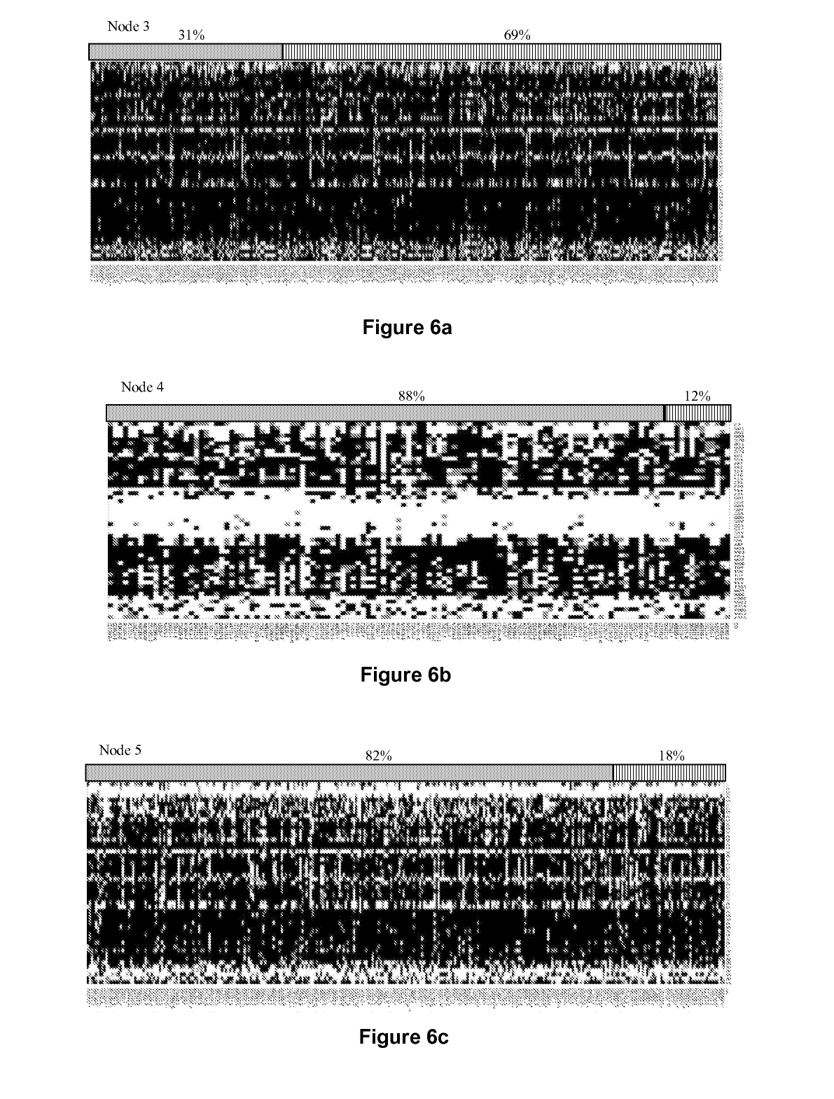

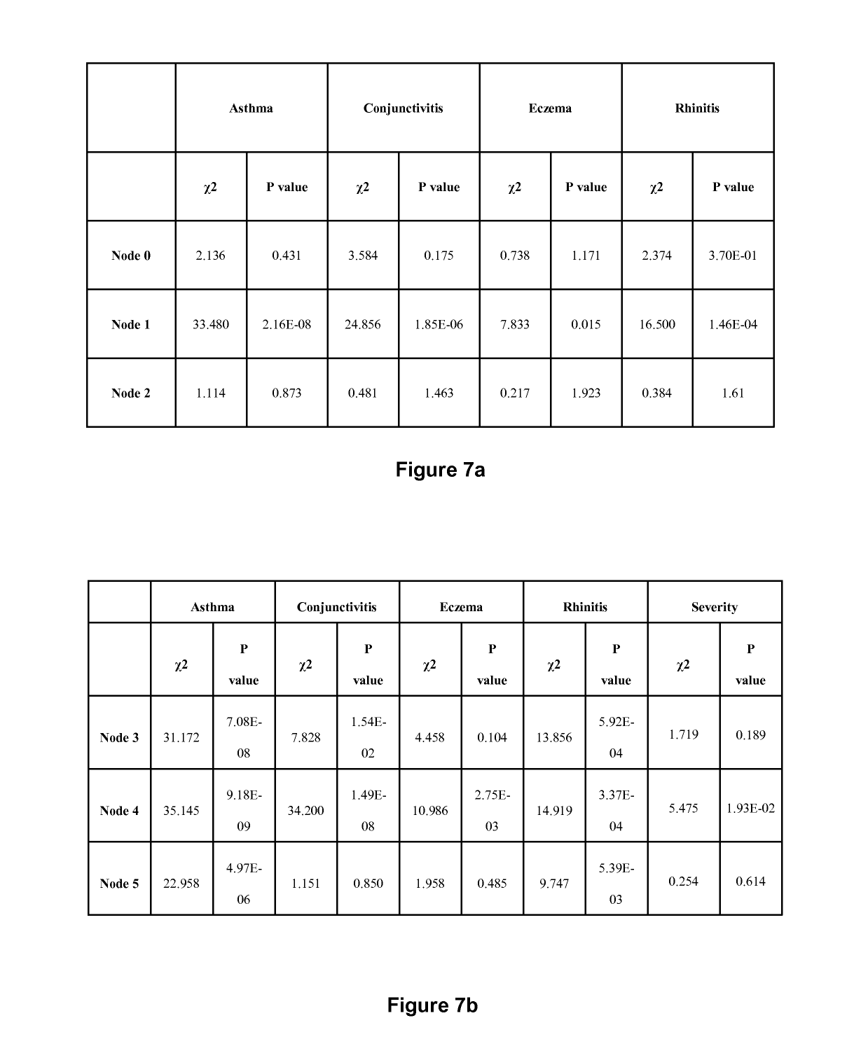

This analysis yielded a list of 51 allergens (Table 4) that were utilized to build a new set of reactivity profiles that were clustered using k-means. The reactivity profiles of nodes 3, 4 and 5 are shown in FIG. 6a, b and c respectively. These new clusters showed some interesting and novel features. Cluster 4 was very similar to cluster 1 in term IgE recognition pattern and sera composition but showed a further increase in the percentage of asthmatic individuals to 88% and a further increase in the statistical significance. These two remaining clusters also showed highly significant statistical differences in their composition. Cluster 3 was enriched in non-asthmatic individuals (69%) while cluster 5 contained a high percentage of asthmatic patients (82%). The distribution of rhinitis and conjunctivitis (but not eczema) in the clusters closely mirrored that of asthma in agreement with other observations that have established a link amongst these atopic diseases. As anticipated we did not observe in cluster 3, 4 and 5 any enrichment in members of the same family nuclei thus indicating that the corresponding profiles are intimately associated with the asthma clinical status rather than to allergen exposure. The two clusters that contained the highest proportion of asthmatic individuals (cluster 4 and 5) were characterized by overlapping IgE recognition profiles. The profiles of cluster 4 showed a specific reactivity against nine additional allergens mainly of food and grasses origin. Notably cluster 4 but not cluster 5 showed a significant association with asthma severity thus unraveling an unsuspected link between disease severity, on one side and the complexity and the specificity of the IgE response on the other one. Further data is presented in FIGS. 7a and b.

Our data demonstrate that associations between asthma and IgE antibody responses to single allergens dramatically underestimate the underlying similarities and differences in individual reactivity to the allergen repertoire that may relevant for understanding the causes, the severity and the progression of the disease. This also explains why making associations between antibody responses and disease hard to identify. On the contrary by analyzing the IgE serum reactivity profile against a large set of allergens we could demonstrate that asthmatic and non-asthmatic individuals differ dramatically in term of number and class of allergens recognized.

### Artificial Neural Network

Professional software applications, which have modules specifically dedicated for ANN, such as the RBF (Radial Basis Function Algorithm-SPSS 17.0.) were utilized for developing the classifier. The RBF is modelled and subjected to supervised training within the SPSS statistics software application-SPSS provides a complete set of powerful functions for training neural networks devoted to classification tasks, such as in our case. The neural network analysis was accomplished by using as input data, a sample data set that includes 51 allergens and the sera reactivity profiles of 827 individuals. Within the sample, 485 are asthma positive individuals, and 342 negative.

To evaluate the actual improvement that was achieved by reducing the number of allergens to be considered by the classifier (by means of the Mann-Whitney test, as illustrated in previously), a separate RBF was trained on the complete set of allergens, and then its results compared with the RBF operating on the filtered set.

The sample was first randomized (see Treatment of the sample section) and the size of the training sample used was about 60% of the entire population, while the remaining individuals were left for validation purposes (testing 10% and holdout of 30%). The training subset was selected using randomization criterion that ensured the representativeness of the sample with respect to the entire population. This was to ensure that the RBF replicates a behaviour that is representative for the whole population. The whole supervised training process was repeated 10 times, each time on a new, previously untrained network, and each time with a new randomised subset of the original population. This was to observe any variation in performance, that may be linked to variability in the representativeness of the training sample.

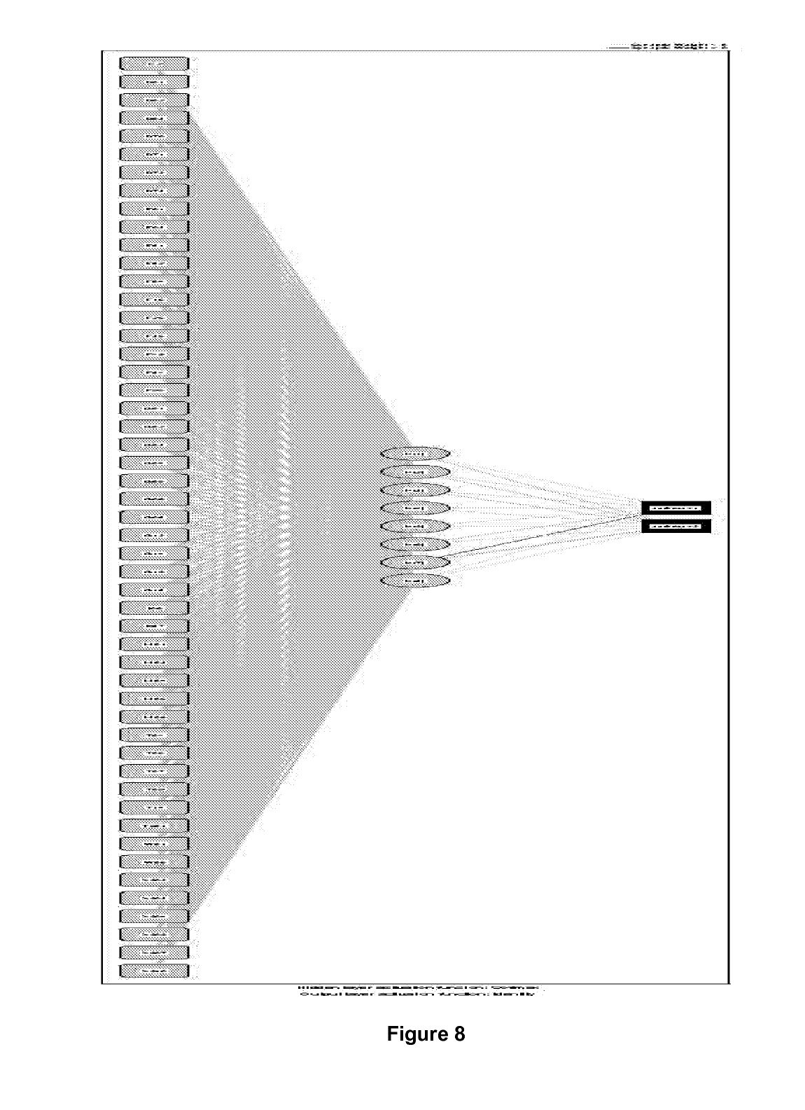

There are three layers in the RBF network (Input, RBF and output layer). There are many types of radial basis functions; we used the Normalized RBF (NRBF). To have an estimate of the real efficiency of the neural network, the neural network was run ten different times. The overall efficiency of the Network is given as the mean of these ten different trials. Asthma was considered as a dependent variable and class score allergens as covariates. The specified relative numbers of case partitions used consisted of training 6 (60%), test 1 (10%), holdout 3 (30%). FIG. 8 illustrates a schematic of the RBF network which consists of three layers: Input (boxes 1-51), hidden (circles 1-8) and output (asthma classes in black boxes) layer respectively.

### Treatment of the Sample Data

To avoid sample order bias, the sera were randomised first. Randomisation was accomplished by using the RAND variable in Matlab, random numbers from 1 to 827 were obtained, and used to flag each sera. An alphabetical order was then used to resample the original sera order.

### Settings Utilised

To have an estimate of the real efficiency of the neural network, the neural network was run ten different times. And the overall efficiency of the Network is given as the mean of these ten different trials.

Analyze, Neural Networks, Radial Basis Function

Variables:

Dependent Variables: asthma

Covariates: C2, D01, D02, D03, D70, D71, D72, D73, E01, E03, E081, E082, F04, F16, F25, F35, F49, F84, F95, G01, G02, G03, G04, G05, G06, G08, G12, G14, G15, G18, I06, K87, M01, M03, M04, M05, M06, T04, T06, T07, T09, T14, T901, W01, W06, X902, X903, X904, X905, X907, X910,

Resealing of Covariates: None

Partitions: Specify relative numbers of cases

training 6 (60%), test 1 (10%), holdout 3 (30%)

### Architecture:

Number of Units in Hidden Layer

Activate Find the best number of units within a range

Range

Activate: Automatically compute range

Activation Function for Hidden Layer

Normalize radial basis function

Overlap Among Hidden Units

Automatically compute the amount of overlap to allow

### Output:

Network Structure

Select: Description, Diagram, Synaptic weights

Network Performance

Select: Model summary, Classification result, Roc curve, Predicted by observed chart

Activate Case processing summary

### Save

Save predicted value or category for each dependent variable

Save predicted pseudo-probability for each dependent variable

Names of Saved Variables

Select Automatically generate unique names

OK

### Export:

Activate: Export synaptic weight estimates to XML file

### Options

User—Missing Values

### Select: Exclude

### Results

A micro-array immunoassay, containing 103 of the most common allergens was used to investigate the IgE serum reactivity of 872 individuals belonging to 283 families in which all the progeny (1 to 3) was affected by asthma.

The individuals enrolled in this study included the two parents and all the descendents (mostly children below the age of 27 (75% of children are below 27). Information concerning a detailed clinical history of asthma (age of onset, severity and persistency) as well as the concomitant presence of other atopic diseases was collected from each individual.

For each serum sample the immunoassay was calibrated to measure the concentration of specific IgE binding to each of the arrayed antigens ranging from 0.35 IU/ml to 100 UI/ml. The reactivity values in UI/ml were converted into class scores using a validated 0-5 scale. This approach generated 872 distinct IgE reactivity profiles and an excess of 90,000 antibody-antigen determinations. A colour-coded digital profile (from black to white) matching the IgE class score (0 to 5) against the arrayed allergens was generated for each serum (FIGS. 5a, b, c and FIGS. 6a, b and c).

The sera had a remarkably heterogeneous IgE reactivity against the arrayed allergens. A number of sera collected from either non-asthmatic parents or asthmatic children reacted with more than 40 allergens though asthmatic individuals showed on average the highest number of individual allergen antibody reactions. To investigate the structure of the reactivity profiles we employed k-means clustering, a partitioning method commonly used to identify group structure within microarray data. The clustering algorithm was run with different values of k (from 3 to 14 and 20) to split the profiles into increasing numbers of clusters. Statistical analysis carried using five specific indicators (Silhouette index, Dunn index, Davies Bouldin, C-index and Isolation index), computed with Machaon, indicated that k=3 is the parameter that by arranging the profiles into three cluster maximizes intra cluster similarities and inter cluster differences.

To look for association between IgE reactivity profiles and asthma we investigated whether the clusters generated at k=3 significantly differed in the frequency of asthmatic and non-asthmatic individuals as well as in the distribution of other traits and pathological conditions (age, sex, conjunctivitis, eczema, rhinitis, asthma persistency and severity). A number of independent statistical analyses (Pearson's X2 and Kruskall-Wallis test) where utilized to assess whether the frequency of each trait and pathological conditions significantly differed between clusters, and between each cluster and the entire sample. In particular, the X2 test was used for analyzing binary attributes (i.e. asthmatic vs non-asthmatic), while the Kruskall-Wallis test was performed on discrete numeric variables (such as the age of asthma onset).

The results of this analysis indicated that the three clusters were significantly different in term of frequency of asthma, conjunctivitis, eczema, rhinitis and sex. Most strikingly cluster 1 showed an impressive (83%) significantly higher proportion (X2==33.480, p=2.16E-08) of asthmatic individuals than the other two other clusters and the entire study sample. The statistical significance was not affected after applying the Bonferroni correction for multiple comparisons.

Similarly significant associations could also be observed for conjunctivitis and rhinitis with cluster 1.

The partitioning of the profiles also highlighted an interesting distribution of the familiar nuclei in the clusters: while clusters 0 and 2 were enriched for members of the same families and showed a similar percentage of asthmatic and non-asthmatic individuals, cluster 1 contained a significantly high proportion of children, but very few parents.

These findings indicate that members of the same families segregating into cluster 0 or 2 had a similar IgE recognition pattern irrespectively of asthma possibly because of the prevailing effect of the exposure to particular set of the arrayed allergens. In contrast, segregation of family members is not observed in cluster 1 as a result of the intimate association of the corresponding IgE seroreactivity profile with the disease.

The array was designed without a detailed knowledge of the allergen exposure of the study population and without any a priori assumption on the role of particular allergens in eliciting an IgE response associated with asthma. It is therefore not surprising that some allergens are rarely recognized while others show similar percentages of reactivity in asthmatic and non-asthmatic individuals.

The reactivity against these allergens contributes to the formation of the profile and could represent a source of “background noise” that masks some associations. To address this problem we attempted to identify in the array the allergens that most contributed to the association with asthma by filtering the results with the Mann-Whitney U test at a threshold of p<0.05 to discard those allergens that did not show differences in the IgE reactivity between asthmatic and non-asthmatic individuals.

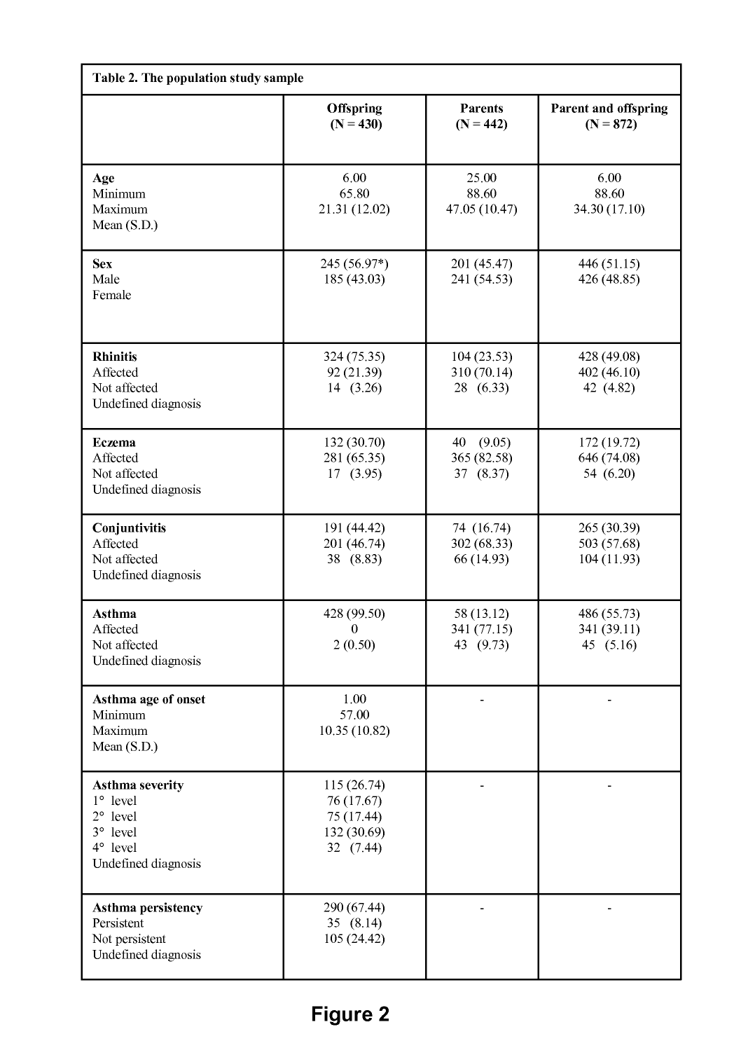

The analysis generated a list of 51 relevant allergens that were utilized to generate new profiles and perform clustering association analysis at k=3. Association studies performed on the new clusters (cluster 3, 4 and 5) revealed a further strengthening of the link between IgE recognition profile and asthma. Cluster 4 showed a high similarity to cluster 1 in term of both its structure and composition though the statistical significance of the association with asthma and the corresponding reactivity profile further increased (X2=35.145, p=9.18E-09), (FIG. 2B). The other two clusters differed substantially from those generated with the complete set of allergens. This time most of the asthmatic individuals that were not included in cluster 1 were significantly associated with the reactivity profile of cluster 5 (X2=22.958, p=4.97E-06) whereas cluster 3 contained most of the non-asthmatic individuals (X2=31.172, p=7.08E-08).

The differences in the distribution of asthmatic and non-asthmatic individuals remained highly significant even after Bonferroni correction for multiple comparisons.

A very strong association was observed in cluster 1 for both conjunctivitis, and rhinitis compared to the other clusters. Notably the clusters generated with the filtered set of allergens did not show a significant co-segregation of family members.

Interestingly two distinct reactivity profiles 4 and 5 were significantly associated with asthma. The two profiles share a common IgE reactivity pattern twenty out of the 51 allergens are recognized by the sera of the two clusters but those of cluster 4 also reacted against nine allergens mainly from the food and grass classes (allergens 19-23 and 27-30).

Notably, cluster 4 showed a significant higher proportion of individuals with diagnosis of severe asthma (severity class 3 and 4) compared to all other clusters and to the population study sample.

### Prediction Utilising Reference Profiles

The unusually strong association linking the reactivity profiles of some clusters and asthma prompted us to generate an artificial neural network (ANN) classifier designed to discriminate between asthmatic and non-asthmatic individuals on the basis of their reactivity profile. This was developed using a radial basis function (RBF) that supports a teaching-by-example training procedure (a.k.a. supervised training/supervised learning).

Each profile example used in the supervised training contained information concerning the reaction values for the 51 filtered allergens, and the health status of the individual respect to the condition of asthma (the expected classification result). The profiles used for training the RBF (the training set) consisted of 60% of the entire study serum samples. An additional 10% of profiles were reserved to assess the predictive accuracy during training (the test set). The remaining individuals, i.e. 30% of the entire population, where left out and used for assessing the performance of the network (the holdout set) in ten independent run. The ANN correctly classified 82% of the asthmatic patients as “asthmatic” and about 72% of the non-asthmatic as “non-asthmatic”. The overall performance of the RBF was consistent with the profile association analysis: the average percentage of asthmatic patients correctly recognized by the RBF classifier as asthmatic is nearly identical to the combined percentage of asthmatic patients present in clusters 4 and 5.

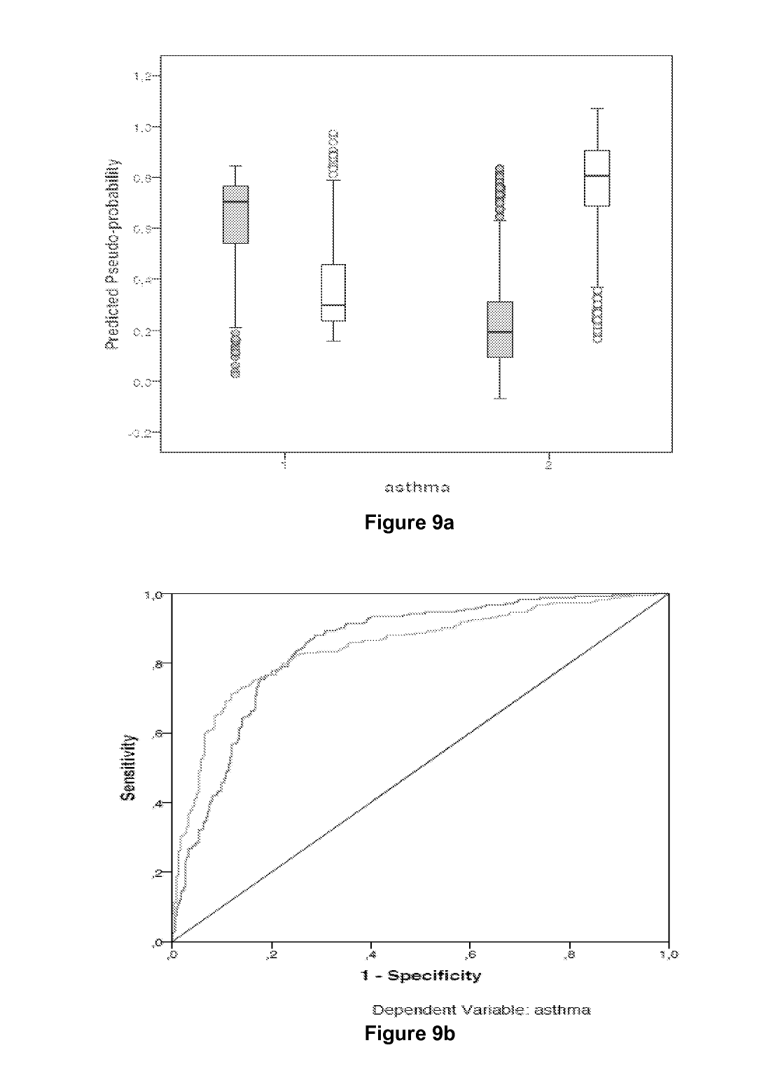

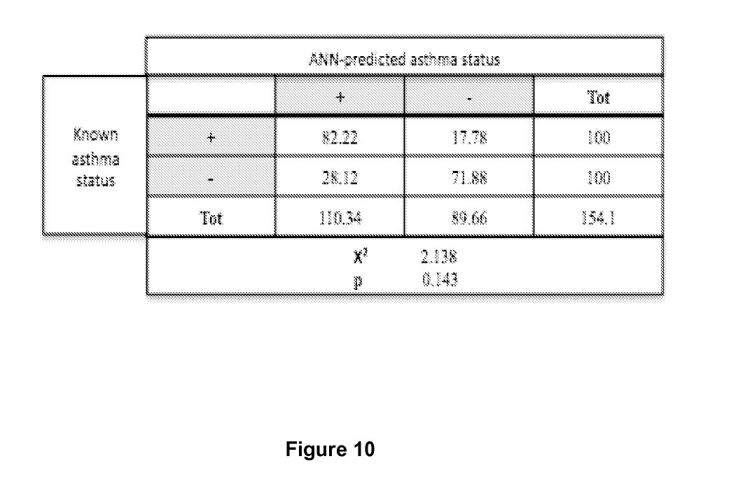

FIG. 9a shows the ANN predicted-by-observed performance chart. The box plots represent the predicted-pseudo-probabilities for the RFB output category; asthma (grey) and non-asthmatic (white) plotted against the known clinical status asthmatic (1) asthmatic (2) for combined training and testing samples. FIG. 9b: The ROC curve calculated on the combined training and testing samples, asthma (black), non-asthmatic (grey). FIG. 10 illustrates ANN asthma classifier consistency performance—The ANN-predicted asthma status of the hold out samples was assessed on a Pearson's X2 test against the known clinical status of the selected individuals.

Construction of Combinatorial Libraries from Asthmatic Patients

To isolate and characterise human IgE antibodies with specificity to the antigens correlated with asthma, conjunctivitis or rhinitis, an IgE combinatorial library was constructed from an asthmatic patient used in the stidy.

Peripheral blood mononuclear cells were obtained from a 150 ml heparinised blood sample obtained from the patient. Briefly, the cells were prepared by Ficoll-Paque density gradient centrifugation.

RNA was prepared by the guanidinium isothiocyanate method of Davis et al., 1986. Several independent cDNA synthesis and PCR amplification reactions were carried out using a RNA PCR kit (Perkin-Elmer).

In brief, the protocol was identical to that used by Steinberger et al., 1996. Total RNA (20-60 μg) was mixed with 10-20 pmol of oligonucleotide primers specific for the constant region of the epsilon chains (C1,5′-GCT ACT AGT TTT GTT GTC GAC CCA GTC; C2,5′-CGA CTG TAA ACT AGT CAC GGT GGG CGG GGT G) and for the light chains (Cκ1a, 5′-GCG CCG TCT AGA ACT AAC ACT CTC CCC TGT TGA AGC TCT TTG TGA CGG GCA AG; Cκ1d, 5′-GCG CCG TCT AGA ATT AAC ACT CTC CCC TGT TGA AGC TCT TTG TGA CGG GCG AAC TCA G; C2,5′-CGC CGT CTA GAA TTA TGA ACA TTC TGT AGG), heated at 65° C. for 5 min and then used in a 2-h reverse transcription reaction according to the suppliers protocol.

The reverse transcription reactions and oligonucleotide primer specific for variable regions of the heavy chains: V, 5′-CAC TCC CAG GTG CAG CTG CTC GAG TCT GG; V, 5′-GTC CTG TCC CAG GTC AAC TTA CTC GAG TCT GG; V, 5′-GTC CAG GTG GAG GTG CAG CTG CTC GAG TCT GG; V, 5′-GTC CTG TCC CAG GTG CAG CTG CTC GAG TCG GG; V, 5′-GTC TGT GCC GAG GTG CAG CTG CTC GAG TCT GG; V, 5′-GTC CTG TCA CAG GTA CAG CTG CTC GAG TCA GG; V, 5′-AG GTG CAG CTG CTC GAG TCT GG; V, 5′-CAG GTG CAG CTG CTC GAG TCG GG; and the κ- or -chains (Vκ1,5′-GAG CCG CAC GAG CCC GAG CTC CAG ATG ACC CAG TCT CC; Vκ1a, 5′-GAC ATC GAG CTC ACC CAG TCT CCA; Vκ2a, 5′-GAG CCG CAC GAG CCC GAG CTC GTG ATG AC(C/T) CAG TCT CC; Vκ3a, 5′-GAA ATT GAG CTC ACG CAG TCT CCA; Vκ3, 5′-GAG CCG CAC GAG CCC GAG CTC GTG (A/T)TG AC(A/G) CAG TCT CC; V1, 5′-AAT TTT GAG CTC ACT CAG CCC CAC; V3, 5′-TCT GTG GAG CTC CAG CCG CCC TCA GTG) were then used in a 100-μl hot start PCR amplification at the following conditions: 1 cycle of 5 min at 95° C. for denaturation, 50 s annealing at 54° C., and 50 s elongation at 72° C. followed by 40 cycles: 1 min denaturation at 92° C., 50 s annealing at 54° C., and 50 s elongation at 72° C. PCR reactions were done using a combination of each constant region and variable region primer and pooled for the construction of the library.

The sequences of oligonucleotide primers of the epsilon chains and light chains were synthesized according to (Kabat et al., 1987). Oligonucleotide primers specific for the variable region of the heavy chains and variable and constant regions of the κ- and -chains were synthesized according to Persson et al. (1991) and Kang et al. (1991b).

### Construction of an IgE Combinatorial Library

The PCR products coding for IgE Fds and light chains were ethanol-precipitated, gel-purified, and cut with SpeI/XhoI and SacI/XbaI, respectively (Boehringer Mannheim). The digested PCR products were ethanol-precipitated and gel-purified.

For the construction of the IgE combinatorial library, light chains were first ligated into the SacI/XbaI site of pComb3H and transformed into Escherichia coli XL-1 Blue to yield a light chain library of 3×10⁷ independent clones.

Plasmid DNA containing the light chain library was then isolated, cut with SpeI/XhoI to release the heavy chain stuffer, and gel-purified. The ligation of the cDNAs coding for the IgE Fds into the light chain plasmid yielded a library of 5×10⁷ independent primary clones.

Molecular biological manipulations used for the construction of the IgE combinatorial library followed the protocols of the Cold Spring Harbor Course on Monoclonal Antibodies from Combinatorial Libraries by Carlos F. Barbas and Dennis R. Burton.

Isolation of Phage Clones Expressing Fab Fragments with Specificity for antigens Correlated with Asthma

ELISA plates (Costar 3690, Cambridge, Mass.) were individually coated with the 51 allergen preparations (0.2 μg/well) that exhibited a correlation with the asthmatic profile. The wells were blocked with phosphate-buffered saline containing 3% (w/v) bovine serum albumin. Freshly prepared phage suspension (approximately 10 plaque-forming units) was added to each well and incubated at room temperature for 2 h. The phage were removed, and the wells were washed with Tris-buffered saline containing 0.05% (v/v) Tween 20 once. Phage were eluted with 0.1 M glycine-HCl, pH 2.2, containing 1 mg/ml bovine serum albumin, and the eluent was neutralized with 2 M Tris. Freshly grown E. coli XL-1 Blue were then infected with the eluted phage. An aliquot was used to determine the titer of infected E. coli. The culture was grown in SB medium containing 50 μg/ml ampicillin and 10 μg/ml tetracyclin. By infection with helper phage VCS M13, filamentous phage were produced for the next round of panning. The panning was repeated four times. During the subsequent pannings, additional washing of the wells was done and individual clones were then analyzed for the production of antigen specific Fabs by ELISA.

Sequence Analysis of the cDNAs Coding for IgE Fds and Light Chains

Clones were checked for the production of antigen specific Fabs by ELISA and for the correct insertion of cDNAs coding for heavy chain fragments and light chains by restriction analysis before sequencing. Plasmid DNA was prepared from recombinant E. coli XL-1 Blue using Qiagen tips (Hilden, Germany). Both DNA strands were sequenced.

Production of Soluble Recombinant Fab Fragments with Antigen Specificity

For the production of soluble Fab fragments, DNA was isolated from several independent clones after the fifth round of panning. The plasmid DNA was digested with SpeI and NheI, recovered from a 1% agarose gel, self-ligated, and retransformed into E. coli XL-1 Blue. E. coli containing the correctly religated plasmid were used to produce soluble Fab fragments.

Briefly, single colonies were inoculated into SB medium containing 20 mM MgCl and 50 μg/ml carbenicillin. The cultures were grown at 37° C. for 6 h and then induced by adding isopropyl-1-thio-β-D-galactopyranoside to a final concentration of 4 mM. Induced E. coli were then grown at 30° C. overnight, and cells were harvested by centrifugation at 3000×g for 10 min at 4° C. The E. coli supernatants were used for ELISA assays, immunoblotting, and for the affinity purification of antigen specific Fabs.

Purification of antigen specific IgE Fabs by Affinity to purified antigens 2.5 mg of purified antigen was coupled to an AminoLink™ column (Pierce) according to the manufacturer's instructions. Approximately 200 ml of E. coli supernatant containing antigen specific Fabs were centrifuged at 20,000×g and subsequently filtered through folded filters (Macherey-Nagel, Duren, Germany) to remove debris from the solution.

The supernatants were applied to the column at 4° C., and the column was then washed extensively with phosphate-buffered saline until no protein could be detected by photometry at 280 nm in the wash fractions. Bound antigen specific Fabs were eluted with 100 mM glycine-HCl, pH 2.7, and neutralized in 3 M Tris, pH 9.

1. A method of identifying antigens associated with asthma, conjunctivitis or rhinitis comprising:
(a) measuring in a first plurality of samples isolated from a group of asymptomatic subjects the levels of IgE reactivity to a plurality of antigens;
(b) measuring in a second plurality of samples isolated from a group of subjects characterised as having asthma, conjunctivitis or rhinitis the levels of IgE reactivity to the plurality of antigens;
(c) identifying a subset of the plurality of antigens which demonstrate significantly different levels of IgE reactivity between the groups of subjects; wherein levels of IgE reactivity to the subset correlate with a clinical diagnosis of asthma, conjunctivitis or rhinitis.

2. The method of claim 1 wherein the levels of reactivity to the plurality of antigens are measured at substantially the same time in each individual sample.

3. The method of claim 2 wherein the first and/or second plurality of samples comprises from 30 to 800 samples.

4. The method of claim 1, wherein the plurality of antigens comprises from 30 to 400 antigens.

5. The methods of claim 4 wherein the antigens are selected as being the most prevalent antigens in a specific region.

6. The method of claim 1, wherein the levels of IgE reactivity are determined by contacting the plurality of antigens with serum isolated from the groups of subjects and determining the amount of IgE bound to each antigen using an anti-IgE antibody.

7. The method of claim 6 wherein the anti-IgE antibody is a labelled antibody.

8. The method of claim 6 wherein the anti-IgE antibody is unlabelled and is detected using a labelled antibody.

9. The method of claim 7 wherein the labelled antibody comprises a fluorescent label.

10. The method of claim 9 wherein the amount of IgE bound to each antigen is determined by fluorescence detection.

11. The method of claim 1 wherein the plurality of antigens are bound to at least one solid support having a plurality of addresses each of which has a distinct antigen disposed thereon.

12. The method of claim 11 wherein the solid support comprises a plurality of separately identifiable beads.

13. The method of claim 11 wherein the solid support is a microarray.

14. The method of claim 13 wherein the antigens have a spotting concentration of from 0.008 mg/ml to 3 mg/ml.

15. A method of assessing if a subject is at risk of developing or has developed asthma, conjunctivitis or rhinitis comprising:
(a) measuring in a sample isolated from said subject the levels of IgE reactivity to a set of biomarkers;
characterised in that the biomarkers are antigens has pre-determined to correlate with a clinical diagnosis of asthma, conjunctivitis or rhinitis and wherein levels of IgE reactivity above 3.51 IU/ml to at least 75% of the set of biomarkers indicates that the subject is likely to develop or has developed asthma, conjunctivitis or rhinitis.

16. The method of claim 15 wherein the set of biomarkers comprises from nine to fifty one antigens.

17. The method of claim 16 wherein the set is selected from the group consisting of antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, 16, K87, M1, M3, M4, M5, M6, T4, T6, T7, T9, T14, T901, W1, W6, X902, X903, X904, X905, X907 and X910.

18. The method of claim 15, wherein
the levels of IgE reactivity are determined by contacting the set of biomarkers with serum isolated from said subject and determining the amount of IgE bound to each antigen using an anti-IgE antibody.

19. The method of claim 18 wherein the anti-IgE antibody is a labelled antibody.

20. The method of claim 18 wherein the anti-IgE antibody is unlabelled and is detected using a labelled antibody.

21. The method of claim 19 wherein the labelled antibody comprises a fluorescent label.

22. The method of claim 21 wherein the amount of IgE bound to each antigen is determined by fluorescence detection.

23. The method of claim 15 wherein the antigens are bound to a solid support.

24. The method of claim 23 wherein the solid support is a microarray.

25. The method of claim 24 wherein the antigens have a spotting concentration of from 0.008 mg/ml to 3 mg/ml.

26. The method of claim 24 wherein the amount of IgE bound to each antigen in the set of biomarkers is determined at substantially the same time.

27. An antigen microarray for use in determining the risk of developing or in the diagnosis of asthma, conjunctivitis or rhinitis the which comprises the antigens F95, G1, G3, G4, G12, G14, G15 and G18 and optionally one or more antigens selected from the group consisting of antigens C2, D1, D2, D3, D70, D71, D72, D73, E1, E3, E81, E82, F4, F16, F25, F35, F49, F84, G2, G5, G6, G8, I6, K87, M1, M3, M4, M5, M6, T4, T6, T7, T9, T14, T901, W1, W6, X902, X903, X904, X905, X907 and X910.

28. A kit comprising:
(i) the antigens F95, G1, G3, G4, G12, G14, G15 and G18;
(ii) optionally one or more of antigens C2, D1, D2, D3, D70, D71, D72, D73, E1, E3, E81, E82, F4, F16, F25, F35, F49, F84, G2, G5, G6, G8, I6, K87, M1, M3, M4, M5, M6, T4, T6, T7, T9, T14, T901, W1, W6, X902, X903, X904, X905, X907 and X910;
(iii) an anti-IgE antibody.

29. The kit of claim 28 comprising antigens C2, D1, D2, D3, D70, D71, D72, D73, E1, E3, E81, E82, F4, F16, F25, F35, F49, F84, F95, G1, G2, G3, G4, G5, G6, G8, G12, G14, G15, G18, 16, K87, M1, M3, M4, M5, M6, T4, T6, T7, T9, T14, T901, W1, W6, X902, X903, X904, X905, X907 and X910 on a microarray.

30. The method of claim 15 for assessing if a subject is at risk of developing or has developed asthma, conjunctivitis or rhinitis.


SEQUENCE LISTING (20 sequences)

SEQ ID NO: 1 - DNA, 27 bp, Artificial
  Primer
gctactagttttgttgtcgacccagtc

SEQ ID NO: 2 - DNA, 31 bp, Artificial
  Primer
cgactgtaaactagtcacggtgggcggggtg

SEQ ID NO: 3 - DNA, 53 bp, Artificial
  Primer
gcgccgtctagaactaacactctcccctgttgaagctctttgtgacgggcaag

SEQ ID NO: 4 - DNA, 58 bp, Artificial
  Primer
gcgccgtctagaattaacactctcccctgttgaagctctttgtgacgggcgaactcag

SEQ ID NO: 5 - DNA, 30 bp, Artificial
  Primer
cgccgtctagaattatgaacattctgtagg

SEQ ID NO: 6 - DNA, 29 bp, Artificial
  Primer
cactcccaggtgcagctgctcgagtctgg

SEQ ID NO: 7 - DNA, 32 bp, Artificial
  Primer
gtcctgtcccaggtcaacttactcgagtctgg

SEQ ID NO: 8 - DNA, 32 bp, Artificial
  Primer
gtccaggtggaggtgcagctgctcgagtctgg

SEQ ID NO: 9 - DNA, 32 bp, Artificial
  Primer
gtcctgtcccaggtgcagctgctcgagtcggg

SEQ ID NO: 10 - DNA, 32 bp, Artificial
  Primer
gtctgtgccgaggtgcagctgctcgagtctgg

SEQ ID NO: 11 - DNA, 32 bp, Artificial
  Primer
gtcctgtcacaggtacagctgctcgagtcagg

SEQ ID NO: 12 - DNA, 22 bp, Artificial
  Primer
aggtgcagctgctcgagtctgg

SEQ ID NO: 13 - DNA, 23 bp, Artificial
  Primer
caggtgcagctgctcgagtcggg

SEQ ID NO: 14 - DNA, 38 bp, Artificial
  Primer
gagccgcacgagcccgagctccagatgacccagtctcc

SEQ ID NO: 15 - DNA, 24 bp, Artificial
  Primer
gacatcgagctcacccagtctcca

SEQ ID NO: 16 - DNA, 38 bp, Artificial
  Primer
gagccgcacgagcccgagctcgtgatgacncagtctcc

SEQ ID NO: 17 - DNA, 24 bp, Artificial
  Primer
gaaattgagctcacgcagtctcca

SEQ ID NO: 18 - DNA, 38 bp, Artificial
  Primer
gagccgcacgagcccgagctcgtgntgacncagtctcc

SEQ ID NO: 19 - DNA, 24 bp, Artificial
  Primer
aattttgagctcactcagccccac

SEQ ID NO: 20 - DNA, 27 bp, Artificial
  Primer
tctgtggagctccagccgccctcagtg